(19) United States
(12) Patent Application Publication
(10) Pub. No.: US 2004/0003424 A1
(43) Pub. Date: Jan. 1, 2004

(54) Transgenic cardiomyocytes with controlled proliferation and differentiation

(75) Inventors: Eric Olson, Dallas, TX (US); Rhonda Bassel-Duby, Dallas, TX (US); David W. Markham, Durham, NC (US); Igor I. Rybkin, Dallas, TX (US); R. Sanders Williams, Durham, NC (US)

Correspondence Address: FULBRIGHT & JAWORSKI L.L.P., SUITE 2400, 600 CONGRESS AVENUE, AUSTIN, TX, 78701-3271, US

(73) Assignee: Board of Regents, The University of Texas System

(21) Appl. No.: 10/379,375

(22) Filed: Mar. 4, 2003

Related U.S. Application Data
(60) Provisional application No. 60/361,521, filed on Mar. 4, 2002.

(51) Int. Cl. A01K 67/027; C12N 5/06

(52) U.S. Cl. 800/18; 800/21; 435/354

(57) ABSTRACT

The present invention provides methods for creating conditionally-immortal cell lines. These transgenic cell lines can be grown indefinitely in culture while maintaining a relatively undifferentiated stated. Upon appropriate switch signal, the cells cease replicating and differentiate much like adult cells. The switch is facilitated by the inactivation of a transforming gene, such as large T antigen. A convenient methodology for such inactivation is Cre-Lox mediated excision of the gene. Cardiac cells are provided as an example of useful a transgenic cell line.

This application claims benefit of U.S. Provisional Serial No. 60/361,521, filed Mar. 4, 2002, the entire contents of which are hereby incorporated by reference.

The government owns rights in the present invention pursuant to grant number RO1-HL61624 from the National Institutes of Health.

### BACKGROUND OF THE INVENTION

1. Field of the Invention

The present invention relates generally to the fields of cellular and molecular biology. More particularly, it concerns the development of transgenic cells engineered to proliferate until given a specific signal to stop proliferating and differentiate into mature cells. The technology is particular important in the study of cell types that are difficult to maintain in a differentiated state in culture.

2. Description of Related Art

Current progress in developmental biology can be greatly attributed to the availability of varieties of cell lines. However, there is a special need for easily accessible cell lines that possess tissue-specific properties. Such cell lines would be valuable tools for studying cell signaling, differential transcriptional programs, and phenotypic changes accompanying normal growth and differentiation. Studies of cardiac development, in particular, have been hampered by the lack of immortalized cell lines capable of proliferation and differentiation.

There have been numerous attempts to derive permanent cell lines from cardiac muscle cells. The major obstacle to this goal is the phenomenon of permanent withdrawal of mammalian cardiac muscle cells from the cell cycle shortly after the birth. Although a small fraction of adult mammalian cardiomyocytes can re-enter the cell cycle and replicate DNA upon physiological or pathological stimulation, there is no significant contribution to cardiac repair by hyperplasia of cardiac cells following damage (i.e., myocardial infarction). Thus, adult cardiomyocytes placed in culture conditions will not divide, and eventually die. Neonatal or embryonic cardiac muscle cells go through limited rounds of cell division in cell culture, but they too ultimately withdraw permanently from the cell cycle.

Such limitations for establishing cell lines from cardiomyocytes leave investigators with several options for the development of cardiac cell lines: 1) isolation of undifferentiated cardioblasts with the ability to differentiate into cardiac muscle cells; 2) conditional selection of a subpopulation of cells from the early cardiac/myogenic embryonic fields that continue to divide in cell culture; 3) development of novel strategies for preventing or reversing irreversible cell cycle withdrawal, based on knowledge of the cardiac cell cycle; and 4) transformation of embryonic or adult cardiac muscle cells by various oncogenic proteins such as Myc, Ras, or SV40 large T-antigen (TAg).

Although cardiac muscle cells can be enriched genetically (Klug et al., 1996) or derived from embryonic stem (ES) cells, teratocarcinoma P19 cells, or blood stem cells, the cell population during the course of differentiation is not homogeneous. Also, cardiomyocytes derived from these sources are altered by prolonged cell culture, and they eventually stop proliferating or become genotypically or phenotypically dissimilar to earlier passages.

Derivation of QCE-6 cells from the precardiac mesoderm of quail or H9c2 cells from embryonic BDIX rat myocardium (Kimes and Brandt, 1976; Brandt et al., 1976) provided useful models for studying early cardiac fate specification or cardiac ion channel function, respectively. However, upon induction of differentiation, QCE-6 cells produce a mixture of cells with limited properties of cardiac or endocardial cells and fail to differentiate into mature cardiomyocytes. On the other hand, H9c2 cells possess properties of cardiac and skeletal muscle cells, expressing a number of muscle specific channels but few structural proteins.

Ectopic expression of various oncogenes such as v-myc and v-Ras (Engelmann et al., 1993) enabled rat embryonic ventricular cardiomyocytes to maintain proliferation with retention of some myocyte characteristics. However, it is unclear whether such cells ultimately produce an immortal cell line.

Promising results have come from the studies utilizing SV40 (TAg) as a transforming factor in murine and human primary cells (Manfredi and Prives, 1998). TAg has been employed in the transformation of heart, skeletal, and smooth muscle cells (Brunskill et al., 2001; Jahn et al., 1996; Morgan et al., 1994; Miller et al., 1994; Tedesco et al., 1995; Parmjit et al., 1991; Gu et al., 1993; Mouly et al., 1996). Each of these myogenic lines showed that TAg could effectively promote proliferation and, in the cases of conditional expression, some degree of differentiation.

AT-1 and HL-1 cell lines were created from the hearts of transgenic mice carrying TAg under the control of the atrial natriuretic factor (ANF) promoter (Kline et al., 1993; Steinhelper et al., 1990). These cell lines exhibited marked capacity for proliferation, at least in the early passages, and expressed many markers specific for heart cells. Some of the cells even possessed spontaneous contractility. However, the potent transforming activity of TAg results in the loss of growth control with consequent abnormalities in cell morphology and gene expression.

Thus, despite these numerous attempts and limited successes, a faithful reproduction of cardiac cell function in the context of a stable cell line has not yet been achieved.

### SUMMARY OF THE INVENTION

Thus, in accordance with the present invention, there is provided a transgenic mouse, cells of which comprise an expression cassette comprising a tissue selective promoter operably linked to a nucleic acid segment encoding SV40 large T antigen, wherein said nucleic acid segment is flanked 5′ and 3′ by site specific excision sequences. The tissue selective promoter may be preferentially active in cardiac cells, such as Nkx2.5. The site specific excision sequences may be loxP sites. The expression cassette may further comprise a selectable or screenable marker.

In another embodiment, there is provided a method for obtaining a transgenic murine progenitor cell line comprising (a) transforming one or more murine embryonic cells with an expression cassette comprising a tissue selective promoter operably linked to a nucleic acid segment encoding SV40 large T antigen, wherein said nucleic acid segment is flanked 5′ and 3′ by site specific excision sequences; (b) inserting said one or more murine embryonic cells into a surrogate mouse mother; (c) obtaining one or more pups from said surrogate mouse mother; (d) identifying one or more pups that express SV40 large T antigen in a tissue selective manner; and (e) obtaining cells from said one or more pups that express SV40 large T antigen. The tissue selective promoter may be preferentially active in cardiac cells, such as Nkx2.5. The site specific excision sequences may be from loxP sites. The expression cassette may further comprise a selectable or screenable marker. The method may further comprise the step of activating site specific excision, thereby eliminating said nucleic acid segment encoding SV40 large T antigen. The step of activating site specific excision may comprise transforming cells of step (e) with an expression construct comprising a promoter operably linked to a nucleic acid segment encoding Cre protein. The expression construct may be a viral expression construct, for example, adenovirus. The promoter may be a constitutive promoter or a tissue selective promoter.

In yet another embodiment, there is provided a transgenic murine progenitor cell line comprising an expression cassette comprising a tissue selective promoter operably linked to a nucleic acid segment encoding SV40 large T antigen, wherein said nucleic acid segment is flanked 5′ and 3′ by site specific excision sequences. The tissue selective promoter may be preferentially active in cardiac cells, such as Nkx2.5. The site specific excision sequences may be loxP sites. The expression cassette further may further comprise a selectable or screenable marker. The cell line may be derived from cells of liver, neuronal, glial, skeletal satellite, cardiac or erythroid tissue.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1—Schematic for methodology.

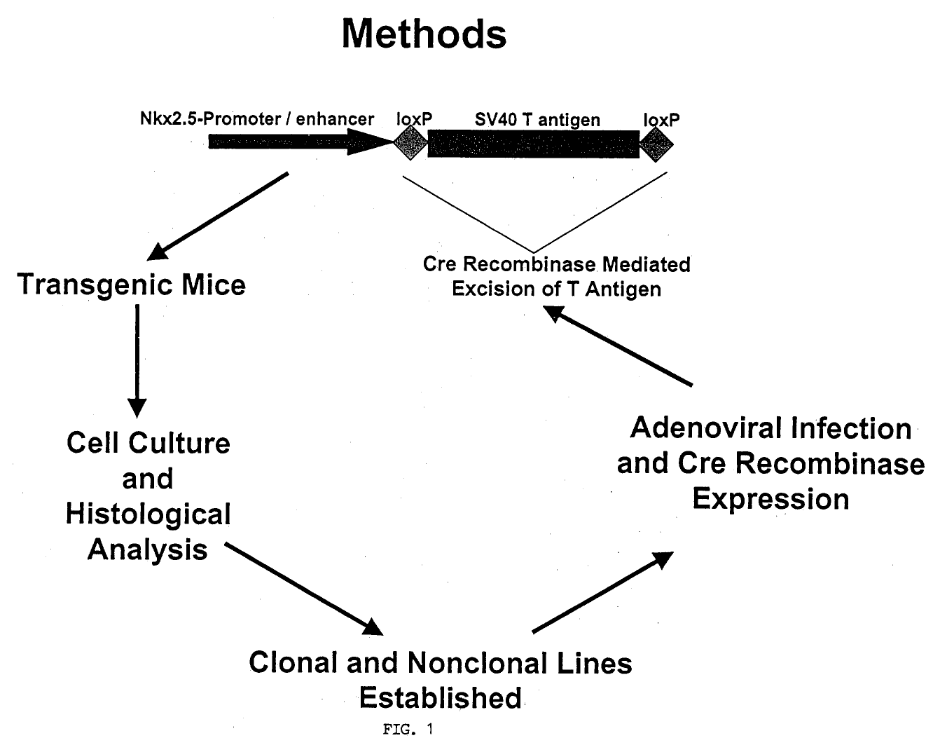

FIG. 2—Nk-TAg transgene and cardiac tumors in Nk-TAg transgenic mouse. A, Diagram of the transgenic construct Nk-TAg containing the Nkx2. 5 cis-regulatory sequences consisting of the early cardiac-specific enhancer region (−9435 to −7353 bp) fused to the endogenous promoter (−265 to +262 bp) linked to the SV-40 large T-antigen coding region.

FIG. 3—Characterization of Nk-TAg cardiac cell lines. Two independent Nk-TAg cell lines (lines 5 and 20) were plated at a low density (1.5×105 cells/100 mm plate) and were counted for five subsequent days. Cells reached confluence on day four but failed to undergo growth arrest.

FIGS. 4A-4C—Analysis of NkL-TAg transgenic mouse cell lines. FIG. 4A, Diagram of NkL-TAg transgenic construct containing the Nkx2.5 cis-regulatory sequences consisting of the early cardiac-specific enhancer region (−9435 to −7353 bp) fused to the endogenous promoter (−265 to +262 bp) linked to the SV-40 large T-antigen coding region flanked by loxP sites (red diamonds). FIG. 4B, BrdU incorporation was measured in NkL-TAg cells (control) and NkL-TAg cells infected with Ad-Cre. Equivalent numbers of cells were counted at 20× magnification. FIG. 4C, NkL-TAg (control) or NkL-TAg cells infected with Ad-LacZ or Ad-Cre were plated at 2×105 cells per 100 mm plate. Cells were counted for 5 subsequent days. NkL-TAg cells stop proliferating in response to excision of the TAg gene.

FIGS. 5A-B—Measurement of calcium transients in NkL-TAg cell lines. FIG. 5A, NkL-TAg cells infected with Ad-Cre were subjected to an electrical stimulation at 1.5 Hz (Ion Optix) with current pulses of 4 msec duration and voltages of 40V. Calcium transients were observed by exciting the fura-2 AM loaded cells with alternating wavelengths of 340 and 380 nm, and recording the emission intensity at 510 nm. Calcium transient data for each myocyte were recorded from a minimum of 12 consecutive stimuli. FIG. 5B, similar measurements were performed on mouse adult cardiomyocytes. Pulses are indicated with arrowheads.

### DESCRIPTION OF ILLUSTRATIVE EMBODIMENTS

During cell growth and development, proliferation and differentiation are tightly controlled. It is a common paradigm that proliferating cells are not fully differentiated. However, when they stop proliferating, differentiation proceeds to produce mature, functional cells. For example, fully differentiated adult mammalian cardiac muscle cells (CMC) do not proliferate in vivo or in vitro, and any cardiac cell loss in adult animal is replaced by connective tissue. The same limitation on cardiomyocyte growth has prevented derivation of cardiac cell lines that can be used for cell cycle and signaling transduction studies.

The link between proliferation and differentiation is particularly important in the heart. Heart muscle cells (cardiomyocytes) do not proliferate after the neonatal period. Thus, heart tissue does not have a mechanism to repair itself following injury. The dilemma of non-proliferating heart cells also applies to laboratory experiments. For example, current experiments performed on cardiomyocytes must be performed on cells newly harvested from laboratory animals. Each experiment requires harvesting fresh cells from animals since heart cells will not proliferate in culture.

While several cardiac cell lines have been derived from transformation with different oncogenes, many such cell lines have a poorly differentiated phenotype. It would be of great interest and utility to provide a variety of cell types that could be propagated indefinitely and then induced to differentiate.

I. The Present Invention

The inventors generated a cardiac cell line from ventricular myocytes of a transgenic mouse. A transgene in which the SV40 Large T-antigen was controlled by the distal cardiac-specific (−9435/−7353) and basal promoter of Nkx2.5 was used to transform mouse embryonic cells. Mice developed multiple subendothelial tumor-like structures protruding into the ventricular chambers. Most of the tumors were localized to the free walls of ventricular chambers and not the septum. The tumor-like structures were dissected and isolated cells plated on fibronectin/gelatin coated dishes.

Eighteen individual clones were established and passaged up to 36 times. These clones expressed numerous cardiac-specific markers including Nkx2.5, GATA4 and MEF2C. However, none of the cell lines was able to contract or exit the cell cycle in response to serum deprivation, although they could be quiesced using inhibitors of DNA synthesis.

Using a different construct, where the Large T-antigen transgene is flanked by loxP sites, additional cell lines were created. When a gene for Cre recombinase was delivered into these cells, facilitating excision of the transgene and loss of Large T-antigen, the cells proliferated more slowly, became much larger, and developed a rod-shaped and often binucleate morphology with visible cross-striations. Thus, elimination of Large T-antigen expression appears to permit a significant degree of cardiomyocyte differentiation in these otherwise immortalized cells.

II. Cell Types

In an exemplified embodiment, transgenic cardiac cell lines are created. However, there a number of other cell types for which cell lines are either not available, or for which the existing cell lines lack appropriate distinguishing characteristics. Other suitable cell types are those which lose their primary characteristics upon transformation into immortalized cells. These include neuronal cells, glial cells, liver cells, skeletal satellite cells and erythroid cells.

III. Cell Specific Promoters

Throughout this application, the term “expression construct” is meant to include any type of genetic construct containing a nucleic acid coding for a gene product in which part or all of the nucleic acid encoding sequence is capable of being transcribed. In such embodiments, the nucleic acid encoding the gene product is under transcriptional control of a promoter. A “promoter” refers to a DNA sequence recognized by the synthetic machinery of the cell, or introduced synthetic machinery, required to initiate the specific transcription of a gene. The phrase “under transcriptional control” means that the promoter is in the correct location and orientation in relation to the nucleic acid to control RNA polymerase initiation and expression of the gene.

The term promoter will be used here to refer to a group of transcriptional control modules that are clustered around the initiation site for RNA polymerase II. Much of the thinking about how promoters are organized derives from analyses of several viral promoters, including those for the HSV thymidine kinase (tk) and SV40 early transcription units. These studies, augmented by more recent work, have shown that promoters are composed of discrete functional modules, each consisting of approximately 7-20 bp of DNA, and containing one or more recognition sites for transcriptional activator or repressor proteins.

At least one module in each promoter functions to position the start site for RNA synthesis. The best known example of this is the TATA box, but in some promoters lacking a TATA box, such as the promoter for the mammalian terminal deoxynucleotidyl transferase gene and the promoter for the SV40 late genes, a discrete element overlying the start site itself helps to fix the place of initiation.

Additional promoter elements regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the tk promoter, the spacing between promoter elements can be increased to 50 bp apart before activity begins to decline. Depending on the promoter, it appears that individual elements can function either co-operatively or independently to activate transcription.

In various embodiments, the human cytomegalovirus (CMV) immediate early gene promoter, the SV40 early promoter, the Rous sarcoma virus long terminal repeat, rat insulin promoter and glyceraldehyde-3-phosphate dehydrogenase can be used to obtain high-level expression of the coding sequence of interest. The use of other viral or mammalian cellular or bacterial phage promoters which are well-known in the art to achieve expression of a coding sequence of interest is contemplated as well, provided that the levels of expression are sufficient for a given purpose.

Of particular interest in tissue specific promoters. For example, muscle specific promoters, and more particularly, cardiac specific promoters, are useful in preparing immortalized cardiac cell lines. These include the myosin light chain-2 promoter (Franz et al., 1994; Kelly et al., 1995), the α actin promoter (Moss et al., 1996), the troponin 1 promoter (Bhavsar et al., 1996); the Na+/Ca2+ exchanger promoter (Barnes et al., 1997), the dystrophin promoter (Kimura et al., 1997), the creatine kinase promoter (Ritchie, 1996), the α7 integrin promoter (Ziober & Kramer, 1996), the brain natriuretic peptide promoter (LaPointe et al., 1996), the αB-crystallin/small heat shock protein promoter (Gopal-Srivastava, 1995), and α myosin heavy chain promoter (Yamauchi-Takihara et al., 1989) and the ANF promoter.

IV. SV40 Large T Antigen

SV40 large T antigen is a 708 amino acid protein that plays an important role in SV40 infection and replication. At least six different post-translational products are known, and diverse activities including DNA binding, DNA unwinding, and DNA-independent ATPase activity have been associated with it. It also binds several other host enzymes and regulatory proteins.

The ATP binding site is located at residues 418-528, a zinc finger domain occurs at residues 302-320, and residues 122-134 constitute a nuclear localization sequence. The vast majority of intracellular large T antigen is nuclear or associated with the nuclear matrix. Oligomerization and phosphorylation are post-translational means for regulating SV40 function. Its primary role is to stimulate transcription, possibly in conjunction with cellular transcription factors such as AP1 and AP2, but it also downregulates SV40 early promoter activity later in infection.

In relation to the present invention, large T antigen also functions as a transforming protein. In certain situations, N-terminal fragments are able to support transformation. Though the present invention exemplifies SV40 large T antigen, polyoma virus large T antigen may be used as an alternative.

V. Cre-Lox

Cre is a 38 kDa recombinase protein from bacteriophage P1 which mediates intramolecular (excisive or inversional) and intermolecular (integrative) site specific recombination between loxP sites (see Sauer, 1993). A loxP site (locus of X-ing over) consists of two 13 bp inverted repeats separated by an 8 bp asymmetric spacer region. One cre gene can be isolated from bacteriophage P1 by methods known in the art, for instance, as disclosed by Abremski et al. (1983), the entire disclosure of which is incorporated herein by reference. U.S. Pat. No. 4,959,317, incorporated by reference, describes the basic Cre-Lox system.

One molecule of Cre binds per inverted repeat, or two Cre molecules line up at one loxP site. The recombination occurs in the asymmetric spacer region. Those 8 bases are also responsible for the directionality of the site. Two loxP sequences in opposite orientation to each other invert the intervening piece of DNA, two sites in direct orientation dictate excision of the intervening DNA between the sites leaving one loxP site behind. This precise removal of DNA can be used to activate or eliminate a transgene.

Lox sites are nucleotide sequences at which the gene product of the Cre recombinase can catalyze a site-specific recombination. A LoxP site is a 34 base pair nucleotide sequence which can be isolated from bacteriophage P1 by methods known in the art. One method for isolating a LoxP site from bacteriophage P1 is disclosed by Hoess et al. (1982), the entire disclosure of which is hereby incorporated herein by reference. As stated above, the LoxP site consists of two 13 base pair inverted repeats separated by an 8 base pair spacer region. The nucleotide sequences of the insert repeats and the spacer region of LoxP are as follows:

ATAACTTCGTATA ATGTATGC TATACGAAGTTAT

Other suitable lox sites include LoxB, LoxL and LoxR sites which are nucleotide sequences isolated from E. coli. These sequences are disclosed and described by Hoess et al. (1982), the entire disclosure of which is hereby incorporated herein by reference. Preferably, the lox site is LoxP or LoxC2. The nucleotide sequences of the insert repeats and the spacer region of LoxC2 are as follows:

ACAACTTCGTATA ATGTATGC TATACGAAGTTAT

Johnson et al., in PCT Application No. WO 93/19172, the entire disclosure of which is hereby incorporated herein by reference, describes phage vectors in which the VH genes are flanked by two loxP sites, one of which is a mutant loxP site (loxP 511) with the G at the seventh position in the spacer region of loxP replaced with an A, which prevents recombination within the vector from merely excising the VH genes. However, two loxP 511 sites can recombine via Cre-mediated recombination and, therefore, can be recombined selectively in the presence of one or more wild-type lox sites. The nucleotide sequences of the insert repeats and the spacer region of loxP 511 as follows:

ATAACTTCGTATA ATGTATAC TATACGAAGTTAT

Lox sites can also be produced by a variety of synthetic techniques which are known in the art. For example, synthetic techniques for producing lox sites are disclosed by Ito et al. (1982) and Ogilvie et al. (1981), the entire disclosures of which are hereby incorporated herein by reference.

VI. Delivery of Nucleic Acids

In accordance with the present invention, nucleic acids are delivered to cells in one of two scenarios. First, in formation of transgenic cardiac cells lines, an expression construct encoding a Large T antigen is transferred into cells to permit their continued proliferation. Second, in certain embodiments, a Cre recombinase is transferred into cells, thereby permitting the excision of the Large T antigen construct, in this case flanked by loxP sites.

There are two generally types of gene transfer—viral and non-viral. Each of these are described below.

1. DNA Delivery Using Viral Vectors

The ability of certain viruses to infect cells and/or enter cells via receptor-mediated endocytosis, and/or to integrate into host cell genome and/or express viral genes stably and/or efficiently have made them attractive candidates for the transfer of foreign genes into mammalian cells. Although some viruses that can accept foreign genetic material are limited in the number of nucleotides they can accommodate or in the range of cells they infect, viruses have been generally successful in effecting gene expression. Different types of viral vectors, and techniques for preparing such, are well known in the art.

A. Adenoviral Vectors

A particular method for delivery of the expression constructs involves the use of an adenovirus expression vector. Although adenovirus vectors are known to have a low capacity for integration into genomic DNA, this feature is counterbalanced by the high efficiency of gene transfer afforded by these vectors. “Adenovirus expression vector” is meant to include those constructs containing adenovirus sequences sufficient to (a) support packaging of the construct and (b) to ultimately express a coding region that has been inserted therein.

The expression vector comprises a genetically engineered form of adenovirus. Knowledge of the genetic organization of adenovirus, a 36 kb, linear, double-stranded DNA virus, allows substitution of large pieces of adenoviral DNA with foreign sequences up to 7 kb (Grunhaus et al., 1992). In contrast to retrovirus, the adenoviral infection of host cells does not result in chromosomal integration because adenoviral DNA can replicate in an episomal manner without potential genotoxicity. Also, adenoviruses are structurally stable, and no genome rearrangement has been detected after extensive amplification.

Adenovirus is particularly suitable for use as a gene transfer vector because of its mid-sized genome, ease of manipulation, high titer, wide target-cell range and high infectivity. Both ends of the viral genome contain 100-200 base pair inverted repeats (ITRs), which are cis elements necessary for viral DNA replication and packaging. The early (E) and/or late (L) regions of the genome contain different transcription units that are divided by the onset of viral DNA replication. The E1 region (E1A and/or E1B) encodes proteins responsible for the regulation of transcription of the viral genome and/or a few cellular genes. The expression of the E2 region (E2A and E2B) results in the synthesis of the proteins for viral DNA replication. These proteins are involved in DNA replication, late gene expression and host cell shut-off (Renan, 1990). The products of the late genes, including the majority of the viral capsid proteins, are expressed only after significant processing of a single primary transcript issued by the major late promoter (MLP). The MLP (located at 16.8 m.u.) is particularly efficient during the late phase of infection, and all the mRNA's issued from this promoter possess a 5′-tripartite leader (TPL) sequence which makes them preferred mRNA's for translation.

In a current system, recombinant adenovirus is generated from homologous recombination between shuttle vector and provirus vector. Due to the possible recombination between two proviral vectors, wild-type adenovirus may be generated from this process. Therefore, it is critical to isolate a single clone of virus from an individual plaque and examine its genomic structure.

Generation and propagation of the current adenovirus vectors, which are replication deficient, depend on a unique helper cell line, designated 293, which was transformed from embryonic kidney cells by Ad5 DNA fragments and constitutively expresses E1 proteins (E1A and E1B; Graham et al., 1977). Since the E3 region is dispensable from the adenovirus genome (Jones and Shenk, 1978), the current adenovirus vectors, with the help of 293 cells, carry foreign DNA in either the E1, the D3 and/or both regions (Graham and Prevec, 1991). Recently, adenoviral vectors comprising deletions in the E4 region have been described (U.S. Pat. No. 5,670,488, incorporated herein by reference).

In nature, adenovirus can package approximately 105% of the wild-type genome (Ghosh-Choudhury et al., 1987), providing capacity for about 2 extra kb of DNA. Combined with the approximately 5.5 kb of DNA that is replaceable in the E1 and E3 regions, the maximum capacity of the current adenovirus vector is under 7.5 kb, and about 15% of the total length of the vector. More than 80% of the adenovirus viral genome remains in the vector backbone.

Racher et al. (1995) disclosed improved methods for culturing 293 cells and propagating adenovirus. In one format, natural cell aggregates are grown by inoculating individual cells into 1 liter siliconized spinner flasks (Techne, Cambridge, UK) containing 100-200 ml of medium. Following stirring at 40 rpm, the cell viability is estimated with trypan blue. In another format, Fibra-Cel microcarriers (Bibby Sterlin, Stone, UK) (5 g/l) is employed as follows. A cell inoculum, resuspended in 5 ml of medium, is added to the carrier (50 ml) in a 250 ml Erlenmeyer flask and left stationary, with occasional agitation, for 1 to 4 h. The medium is then replaced with 50 ml of fresh medium and shaking initiated. For virus production, cells are allowed to grow to about 80% confluence, after which time the medium is replaced (to 25% of the final volume) and adenovirus added at an MOI of 0.05. Cultures are left stationary overnight, following which the volume is increased to 100% and shaking commenced for another 72 h.

Other than the requirement that the adenovirus vector be replication defective, and at least conditionally defective, the nature of the adenovirus vector is not believed to be crucial to the successful practice of the invention. The adenovirus may be of any of the 42 different known serotypes and subgroups A-F. Adenovirus type 5 of subgroup C is the preferred starting material in order to obtain the conditional replication-defective adenovirus vector for use in the present invention. This is because Adenovirus type 5 is a adenovirus about which a great deal of biochemical and genetic information is known, and it has historically been used for most constructions employing adenovirus as a vector.

As stated above, the typical vector according to the present invention is replication defective and will not have an adenovirus E1 region. Thus, it will be most convenient to introduce the transforming construct at the position from which the E1-coding sequences have been removed. However, the position of insertion of the construct within the adenovirus sequences is not critical to the invention. The polynucleotide encoding the gene of interest may also be inserted in lieu of the deleted E3 region in E3 replacement vectors as described by Karlsson et al. (1986) and in the E4 region where a helper cell line and helper virus complements the E4 defect.

Adenovirus growth and manipulation is known to those of skill in the art, and exhibits broad host range in vitro and in vivo. This group of viruses can be obtained in high titers, e.g., 10⁹ to 10¹¹ plaque-forming units per ml, and they are highly infective. The life cycle of adenovirus does not require integration into the host cell genome. The foreign genes delivered by adenovirus vectors are episomal and, therefore, have low genotoxicity to host cells. No side effects have been reported in studies of vaccination with wild-type adenovirus (Couch et al., 1963; Top et al., 1971), demonstrating their safety and therapeutic potential as in vivo gene transfer vectors.

Adenovirus vectors have been used in eukaryotic gene expression (Levrero et al., 1991; Gomez-Foix et al., 1992) and vaccine development (Grunhaus et al., 1992; Graham and Prevec, 1992). Recently, animal studies suggested that recombinant adenovirus could be used for gene therapy (Stratford-Perricaudet and Perricaudet, 1991; Stratford-Perricaudet et al., 1990; Rich et al., 1993). Studies in administering recombinant adenovirus to different tissues include trachea instillation (Rosenfeld et al., 1991; Rosenfeld et al., 1992), muscle injection (Ragot et al., 1993), peripheral intravenous injections (Herz and Gerard, 1993) and stereotactic inoculation into the brain (Le Gal La Salle et al., 1993). Recombinant adenovirus and adeno-associated virus (see below) can both infect and transduce non-dividing primary cells.

B. AAV Vectors

Adeno-associated virus (AAV) is an attractive vector system for use in the cell transduction of the present invention as it has a high frequency of integration and it can infect nondividing cells, thus making it useful for delivery of genes into cells, for example, in tissue culture (Muzyczka, 1992) and in vivo. AAV has a broad host range for infectivity (Tratschin et al., 1984; Laughlin et al., 1986; Lebkowski et al., 1988; McLaughlin et al., 1988). Details concerning the generation and use of rAAV vectors are described in U.S. Pat. No. 5,139,941 and U.S. Pat. No. 4,797,368, each incorporated herein by reference.

Studies demonstrating the use of AAV in gene delivery include LaFace et al. (1988); Zhou et al. (1993); Flotte et al. (1993); and Walsh et al. (1994). Recombinant AAV vectors have been used successfully for in vitro and in vivo transduction of marker genes (Kaplitt et al., 1994; Lebkowski et al., 1988; Samulski et al., 1989; Yoder et al., 1994; Zhou et al., 1994; Hermonat and Muzyczka, 1984; Tratschin et al., 1985; McLaughlin et al., 1988) and genes involved in various diseases (Flotte et al., 1992; Ohi et al., 1990; Walsh et al., 1994; Wei et al., 1994). Recently, an AAV vector has been approved for phase I trials for the treatment of cystic fibrosis.

AAV is a dependent parvovirus in that it requires coinfection with another virus (either adenovirus or a member of the herpesvirus family) to undergo a productive infection in cultured cells (Muzyczka, 1992). In the absence of coinfection with helper virus, the wild type AAV genome integrates through its ends into chromosome 19 where it resides in a latent state as a provirus (Kotin et al., 1990; Samulski et al., 1991). rAAV, however, is not restricted to chromosome 19 for integration unless the AAV Rep protein is also expressed (Shelling and Smith, 1994). When a cell carrying an AAV provirus is superinfected with a helper virus, the AAV genome is “rescued” from the chromosome or from a recombinant plasmid, and a normal productive infection is established (Samulski et al., 1989; McLaughlin et al., 1988; Kotin et al., 1990; Muzyczka, 1992).

Typically, recombinant AAV (rAAV) virus is made by cotransfecting a plasmid containing the gene of interest flanked by the two AAV terminal repeats (McLaughlin et al., 1988; Samulski et al., 1989; each incorporated herein by reference) and an expression plasmid containing the wild-type AAV coding sequences without the terminal repeats, for example pIM45 (McCarty et al., 1991; incorporated herein by reference). The cells are also infected or transfected with adenovirus or plasmids carrying the adenovirus genes required for AAV helper function. rAAV virus stocks made in such fashion are contaminated with adenovirus which must be physically separated from the rAAV particles (for example, by cesium chloride density centrifugation). Alternatively, adenovirus vectors containing the AAV coding regions or cell lines containing the AAV coding regions and some or all of the adenovirus helper genes could be used (Yang et al., 1994; Clark et al., 1995). Cell lines carrying the rAAV DNA as an integrated provirus can also be used (Flotte et al., 1995).

C. Retroviral Vectors

Retroviruses have promise as gene delivery vectors due to their ability to integrate their genes into the host genome, transferring a large amount of foreign genetic material, infecting a broad spectrum of species or cell types and of being packaged in special cell-lines (Miller, 1992).

The retroviruses are a group of single-stranded RNA viruses characterized by an ability to convert their RNA to double-stranded DNA in infected cells by a process of reverse-transcription (Coffin, 1990). The resulting DNA then stably integrates into cellular chromosomes as a provirus and directs synthesis of viral proteins. The integration results in the retention of the viral gene sequences in the recipient cell and its descendants. The retroviral genome contains three genes, gag, pol, and env that code for capsid proteins, polymerase enzyme, and envelope components, respectively. A sequence found upstream from the gag gene contains a signal for packaging of the genome into virions. Two long terminal repeat (LTR) sequences are present at the 5′ and 3′ ends of the viral genome. These contain strong promoter and enhancer sequences and are also required for integration in the host cell genome (Coffin, 1990).

In order to construct a retroviral vector, a nucleic acid encoding a gene of interest is inserted into the viral genome in the place of certain viral sequences to produce a virus that is replication-defective. In order to produce virons, a packaging cell line containing the gag, pol, and env genes but without the LTR and packaging components is constructed (Mann et al., 1983). When a recombinant plasmid containing a cDNA, together with the retroviral LTR and packaging sequences is introduced into this cell line (by calcium phosphate precipitation for example), the packaging sequence allows the RNA transcript of the recombinant plasmid to be packaged into viral particles, which are then secreted into the culture media (Nicolas and Rubenstein, 1988; Temin, 1986; Mann et al., 1983). The media containing the recombinant retroviruses is then collected, optionally concentrated, and used for gene transfer. Retroviral vectors are able to infect a broad variety of cell types. However, integration and stable expression require the division of host cells (Paskind et al., 1975).

Concern with the use of defective retrovirus vectors is the potential appearance of wild-type replication-competent virus in the packaging cells. This can result from recombination events in which the intact sequence from the recombinant virus inserts upstream from the gag, pol, env sequence integrated in the host cell genome. However, new packaging cell lines are now available that should greatly decrease the likelihood of recombination (Markowitz et al., 1988; Hersdorffer et al., 1990).

Gene delivery using second generation retroviral vectors has been reported. Kasahara et al (1994) prepared an engineered variant of the Moloney murine leukemia virus, that normally infects only mouse cells, and modified an envelope protein so that the virus specifically bound to, and infected, cells bearing the erythropoietin (EPO) receptor. This was achieved by inserting a portion of the EPO sequence into an envelope protein to create a chimeric protein with a new binding specificity.

D. Other Viral Vectors

Other viral vectors may be employed as expression constructs in the present invention. Vectors derived from viruses such as vaccinia virus (Ridgeway, 1988; Baichwal and Sugden, 1986; Coupar et al., 1988), sindbis virus, cytomegalovirus or herpes simplex virus may be employed. They offer several attractive features for various cells (Friedmann, 1989; Ridgeway, 1988; Baichwal and Sugden, 1986; Coupar et al., 1988; Horwich et al., 1990).

With the recent recognition of defective hepatitis B viruses, new insight was gained into the structure-function relationship of different viral sequences. In vitro studies showed that the virus could retain the ability for helper-dependent packaging and reverse transcription despite the deletion of up to 80% of its genome (Horwich et al., 1990). This suggested that large portions of the genome could be replaced with foreign genetic material. Chang et al. recently introduced the chloramphenicol acetyltransferase (CAT) gene into duck hepatitis B virus genome in the place of the polymerase, surface, and pre-surface coding sequences. It was cotransfected with wild-type virus into an avian hepatoma cell line. Culture media containing high titers of the recombinant virus were used to infect primary duckling hepatocytes. Stable CAT gene expression was detected for at least 24 days after transfection (Chang et al., 1991).

In certain further embodiments, the gene therapy vector will be HSV. A factor that makes HSV an attractive vector is the size and organization of the genome. Because HSV is large, incorporation of multiple genes and expression cassettes is less problematic than in other smaller viral systems. In addition, the availability of different viral control sequences with varying performance (temporal, strength, etc.) makes it possible to control expression to a greater extent than in other systems. It also is an advantage that the virus has relatively few spliced messages, further easing genetic manipulations. HSV also is relatively easy to manipulate and can be grown to high titers. Thus, delivery is less of a problem, both in terms of volumes needed to attain sufficient MOI and in a lessened need for repeat dosings.

E. Modified Viruses

In still further embodiments of the present invention, the nucleic acids to be delivered are housed within an infective virus that has been engineered to express a specific binding ligand. The virus particle will thus bind specifically to the cognate receptors of the target cell and deliver the contents to the cell. A novel approach designed to allow specific targeting of retrovirus vectors was recently developed based on the chemical modification of a retrovirus by the chemical addition of lactose residues to the viral envelope. This modification can permit the specific infection of hepatocytes via sialoglycoprotein receptors.

Another approach to targeting of recombinant retroviruses was designed in which biotinylated antibodies against a retroviral envelope protein or against a specific cell receptor were used. The antibodies were coupled via the biotin components by using streptavidin (Roux et al., 1989). Using antibodies against major histocompatibility complex class I or class II antigens, they demonstrated the infection of a variety of cells that bore those surface antigens with an ecotropic virus in vitro (Roux et al., 1989).

2. Non-Viral Transformation

Suitable methods for non-viral nucleic acid delivery for transformation of a cell for use with the current invention are believed to include virtually any method by which a nucleic acid (e.g., DNA) as would be known to one of ordinary skill in the art. Such methods include, but are not limited to, direct delivery of DNA such as by injection (U.S. Pat. Nos. 5,994,624, 5,981,274, 5,945,100, 5,780,448, 5,736,524, 5,702,932, 5,656,610, 5,589,466 and 5,580,859, each incorporated herein by reference), including microinjection (Harlan and Weintraub, 1985; U.S. Pat. No. 5,789,215, incorporated herein by reference); by electroporation (U.S. Pat. No. 5,384,253, incorporated herein by reference; Tur-Kaspa et al., 1986; Potter et al., 1984); by calcium phosphate precipitation (Graham and Van Der Eb, 1973; Chen and Okayama, 1987; Rippe et al., 1990); by using DEAE-dextran followed by polyethylene glycol (Gopal, 1985); by direct sonic loading (Fechheimer et al., 1987); by liposome mediated transfection (Nicolau and Sene, 1982; Fraley et al., 1979; Nicolau et al., 1987; Wong et al., 1980; Kaneda et al., 1989; Kato et al., 1991) and receptor-mediated transfection (Wu and Wu, 1987; Wu and Wu, 1988); by microprojectile bombardment (PCT Application Nos. WO 94/09699 and 95/06128; U.S. Pat. Nos. 5,610,042; 5,322,783 5,563,055, 5,550,318, 5,538,877 and 5,538,880, and each incorporated herein by reference); by agitation with silicon carbide fibers (U.S. Pat. Nos. 5,302,523 and 5,464,765, each incorporated herein by reference); by PEG-mediated transformation of protoplasts (Omirulleh et al., 1993; U.S. Pat. Nos. 4,684,611 and 4,952,500, each incorporated herein by reference); by desiccation/inhibition-mediated DNA uptake (Potrykus et al., 1985), and any combination of such methods.

A. Injection

In certain embodiments, a nucleic acid may be delivered to an organelle, a cell, a tissue or an organism via one or more injections (i.e., a needle injection), such as, for example, subcutaneously, intradermally, intramuscularly, intravenously, intraperitoneally, etc. Methods of injection of vaccines are well known to those of ordinary skill in the art (e.g., injection of a composition comprising a saline solution). Further embodiments of the present invention include the introduction of a nucleic acid by direct microinjection. Direct microinjection has been used to introduce nucleic acid constructs into Xenopus oocytes (Harland and Weintraub, 1985). The amount of DNA used may vary upon the nature of the antigen as well as the organelle, cell, tissue or organism used

B. E1 ectroporation

In certain embodiments of the present invention, a nucleic acid is introduced into an organelle, a cell, a tissue or an organism via electroporation. E1 ectroporation involves the exposure of a suspension of cells and DNA to a high-voltage electric discharge. In some variants of this method, certain cell wall-degrading enzymes, such as pectin-degrading enzymes, are employed to render the target recipient cells more susceptible to transformation by electroporation than untreated cells (U.S. Pat. No. 5,384,253, incorporated herein by reference). Alternatively, recipient cells can be made more susceptible to transformation by mechanical wounding.

Transfection of eukaryotic cells using electroporation has been quite successful. Mouse pre-B lymphocytes have been transfected with human kappa-immunoglobulin genes (Potter et al., 1984), and rat hepatocytes have been transfected with the chloramphenicol acetyltransferase gene (Tur-Kaspa et al., 1986) in this manner.

C. Calcium Phosphate

In other embodiments of the present invention, a nucleic acid is introduced to the cells using calcium phosphate precipitation. Human KB cells have been transfected with adenovirus 5 DNA (Graham and Van Der Eb, 1973) using this technique. Also in this manner, mouse L(A9), mouse C127, CHO, CV-1, BHK, NIH3T3 and HeLa cells were transfected with a neomycin marker gene (Chen and Okayama, 1987), and rat hepatocytes were transfected with a variety of marker genes (Rippe et al., 1990).

D. DEAE-Dextran

In another embodiment, a nucleic acid is delivered into a cell using DEAE-dextran followed by polyethylene glycol. In this manner, reporter plasmids were introduced into mouse myeloma and erythroleukemia cells (Gopal, 1985).

E. Sonication Loading

Additional embodiments of the present invention include the introduction of a nucleic acid by direct sonic loading. LTK-fibroblasts have been transfected with the thymidine kinase gene by sonication loading (Fechheimer et al., 1987).

F. Liposome-Mediated Transfection

In a further embodiment of the invention, a nucleic acid may be entrapped in a lipid complex such as, for example, a liposome. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh and Bachhawat, 1991). Also contemplated is an nucleic acid complexed with Lipofectamine (Gibco BRL) or Superfect (Qiagen).

Liposome-mediated nucleic acid delivery and expression of foreign DNA in vitro has been very successful (Nicolau and Sene, 1982; Fraley et al., 1979; Nicolau et al., 1987). The feasibility of liposome-mediated delivery and expression of foreign DNA in cultured chick embryo, HeLa and hepatoma cells has also been demonstrated (Wong et al., 1980).

In certain embodiments of the invention, a liposome may be complexed with a hemagglutinating virus (HVJ). This has been shown to facilitate fusion with the cell membrane and promote cell entry of liposome-encapsulated DNA (Kaneda et al., 1989). In other embodiments, a liposome may be complexed or employed in conjunction with nuclear non-histone chromosomal proteins (HMG-1) (Kato et al., 1991). In yet further embodiments, a liposome may be complexed or employed in conjunction with both HVJ and HMG-1. In other embodiments, a delivery vehicle may comprise a ligand and a liposome.

G. Receptor Mediated Transfection

Still further, a nucleic acid may be delivered to a target cell via receptor-mediated delivery vehicles. These take advantage of the selective uptake of macromolecules by receptor-mediated endocytosis that will be occurring in a target cell. In view of the cell type-specific distribution of various receptors, this delivery method adds another degree of specificity to the present invention.

Certain receptor-mediated gene targeting vehicles comprise a cell receptor-specific ligand and a nucleic acid-binding agent. Others comprise a cell receptor-specific ligand to which the nucleic acid to be delivered has been operatively attached. Several ligands have been used for receptor-mediated gene transfer (Wu and Wu, 1987; Wagner et al., 1990; Perales et al., 1994; Myers, EPO 0273085), which establishes the operability of the technique. Specific delivery in the context of another mammalian cell type has been described (Wu and Wu, 1993; incorporated herein by reference). In certain aspects of the present invention, a ligand will be chosen to correspond to a receptor specifically expressed on the target cell population.

In other embodiments, a nucleic acid delivery vehicle component of a cell-specific nucleic acid targeting vehicle may comprise a specific binding ligand in combination with a liposome. The nucleic acid(s) to be delivered are housed within the liposome and the specific binding ligand is functionally incorporated into the liposome membrane. The liposome will thus specifically bind to the receptor(s) of a target cell and deliver the contents to a cell. Such systems have been shown to be functional using systems in which, for example, epidermal growth factor (EGF) is used in the receptor-mediated delivery of a nucleic acid to cells that exhibit upregulation of the EGF receptor.

In still further embodiments, the nucleic acid delivery vehicle component of a targeted delivery vehicle may be a liposome itself, which will preferably comprise one or more lipids or glycoproteins that direct cell-specific binding. For example, lactosyl-ceramide, a galactose-terminal asialganglioside, have been incorporated into liposomes and observed an increase in the uptake of the insulin gene by hepatocytes (Nicolau et al., 1987). It is contemplated that the tissue-specific transforming constructs of the present invention can be specifically delivered into a target cell in a similar manner.

H. Microprojectile Bombardment

Microprojectile bombardment techniques can be used to introduce a nucleic acid into a cell, tissue or organism (U.S. Pat. No. 5,550,318; U.S. Pat. No. 5,538,880; U.S. Pat. No. 5,610,042; and PCT Application WO 94/09699; each of which is incorporated herein by reference). This method depends on the ability to accelerate DNA-coated microprojectiles to a high velocity allowing them to pierce cell membranes and enter cells without killing them (Klein et al., 1987). There are a wide variety of microprojectile bombardment techniques known in the art, many of which are applicable to the invention.

In this microprojectile bombardment, one or more particles may be coated with at least one nucleic acid and delivered into cells by a propelling force. Several devices for accelerating small particles have been developed. One such device relies on a high voltage discharge to generate an electrical current, which in turn provides the motive force (Yang et al., 1990). The microprojectiles used have consisted of biologically inert substances such as tungsten or gold particles or beads. Exemplary particles include those comprised of tungsten, platinum, and preferably, gold. It is contemplated that in some instances DNA precipitation onto metal particles would not be necessary for DNA delivery to a recipient cell using microprojectile bombardment. However, it is contemplated that particles may contain DNA rather than be coated with DNA. DNA-coated particles may increase the level of DNA delivery via particle bombardment but are not, in and of themselves, necessary.

For the bombardment, cells in suspension are concentrated on filters or solid culture medium. Alternatively, immature embryos or other target cells may be arranged on solid culture medium. The cells to be bombarded are positioned at an appropriate distance below the macroprojectile stopping plate.

An illustrative embodiment of a method for delivering DNA into a cell (e.g., a plant cell) by acceleration is the Biolistics Particle Delivery System, which can be used to propel particles coated with DNA or cells through a screen, such as a stainless steel or Nytex screen, onto a filter surface covered with cells, such as for example, a monocot plant cells cultured in suspension. The screen disperses the particles so that they are not delivered to the recipient cells in large aggregates. It is believed that a screen intervening between the projectile apparatus and the cells to be bombarded reduces the size of projectiles aggregate and may contribute to a higher frequency of transformation by reducing the damage inflicted on the recipient cells by projectiles that are too large.

VII. Transgenic Animals

Transgenic non-human animals (e.g., mammals) of the invention can be of a variety of species including murine (rodents; e.g., mice, rats), avian (chicken, turkey, fowl), bovine (beef, cow, cattle), ovine (lamb, sheep, goats), porcine (pig, swine), and piscine (fish). In a preferred embodiment, the transgenic animal is a rodent, such as a mouse or a rat. Detailed methods for generating non-human transgenic animals are described herein. Transgenic gene constructs can be introduced into the germ line of an animal to make a transgenic mammal. For example, one or several copies of the construct may be incorporated into the genome of a mammalian embryo by standard transgenic techniques.

In an exemplary embodiment, the “transgenic non-human animals” of the invention are produced by introducing transgenes into the germline of the non-human animal. Embryonal target cells at various developmental stages can be used to introduce transgenes. Different methods are used depending on the stage of development of the embryonal target cell. The specific line(s) of any animal used to practice this invention are selected for general good health, good embryo yields, good pronuclear visibility in the embryo, and good reproductive fitness. In addition, the haplotype is a significant factor.

Introduction of the transgene into the embryo can be accomplished by any means known in the art such as, for example, microinjection, electroporation, or lipofection. For example, the Fc receptor transgene can be introduced into a mammal by microinjection of the construct into the pronuclei of the fertilized mammalian egg(s) to cause one or more copies of the construct to be retained in the cells of the developing mammal(s). Following introduction of the transgene construct into the fertilized egg, the egg may be incubated in vitro for varying amounts of time, or reimplanted into the surrogate host, or both. Reimplantation is accomplished using standard methods. Usually, the surrogate host is anesthetized, and the embryos are inserted into the oviduct. The number of embryos implanted into a particular host will vary by species, but will usually be comparable to the number of off spring the species naturally produces. In vitro incubation to maturity is within the scope of this invention. One common method in to incubate the embryos in vitro for about 1-7 days, depending on the species, and then reimplant them into the surrogate host.

The progeny of the transgenically manipulated embryos can be tested for the presence of the construct by Southern blot analysis of the segment of tissue. The litters of transgenically altered mammals can be assayed after birth for the incorporation of the construct into the genome of the offspring. Preferably, this assay is accomplished by hybridizing a probe corresponding to the DNA sequence coding for the desired recombinant protein product or a segment thereof onto chromosomal material from the progeny. Those mammalian progeny found to contain at least one copy of the construct in their genome are grown to maturity.

For the purposes of this invention a zygote is essentially the formation of a diploid cell which is capable of developing into a complete organism. Generally, the zygote will be comprised of an egg containing a nucleus formed, either naturally or artificially, by the fusion of two haploid nuclei from a gamete or gametes. Thus, the gamete nuclei must be ones which are naturally compatible, i.e., ones which result in a viable zygote capable of undergoing differentiation and developing into a functioning organism. Generally, a euploid zygote is preferred. If an aneuploid zygote is obtained, then the number of chromosomes should not vary by more than one with respect to the euploid number of the organism from which either gamete originated.

In addition to similar biological considerations, physical ones also govern the amount (e.g., volume) of exogenous genetic material which can be added to the nucleus of the zygote or to the genetic material which forms a part of the zygote nucleus. If no genetic material is removed, then the amount of exogenous genetic material which can be added is limited by the amount which will be absorbed without being physically disruptive. Generally, the volume of exogenous genetic material inserted will not exceed about 10 picoliters. The physical effects of addition must not be so great as to physically destroy the viability of the zygote. The biological limit of the number and variety of DNA sequences will vary depending upon the particular zygote and functions of the exogenous genetic material and will be readily apparent to one skilled in the art, because the genetic material, including the exogenous genetic material, of the resulting zygote must be biologically capable of initiating and maintaining the differentiation and development of the zygote into a functional organism.

Transgenic offspring of the surrogate host may be screened for the presence and/or expression of the transgene by any suitable method. Screening is often accomplished by Southern blot or Northern blot analysis, using a probe that is complementary to at least a portion of the transgene. Western blot analysis using an antibody against the protein encoded by the transgene may be employed as an alternative or additional method for screening for the presence of the transgene product. Typically, DNA is prepared from tail tissue and analyzed by Southern analysis or PCR for the transgene. Alternatively, the tissues or cells believed to express the transgene at the highest levels are tested for the presence and expression of the transgene using Southern analysis or PCR, although any tissues or cell types may be used for this analysis.

Alternative or additional methods for evaluating the presence of the transgene include, without limitation, suitable biochemical assays such as enzyme or immunological assays, histological stains for particular marker or enzyme activities, flow cytometric analysis, and the like. Analysis of the blood may also be useful to detect the presence of the transgene product in the blood, as well as to evaluate the effect of the transgene on the levels of various types of blood cells and other blood constituents.

Progeny of the transgenic animals may be obtained by mating the transgenic animal with a suitable partner, or by in vitro fertilization of eggs and sperm obtained from the transgenic animal. Where mating with a partner is to be performed, the partner may or may not be transgenic or a knockout; where it is transgenic, it may contain the same or a different transgene, or both. Alternatively, the partner may be a parental line. Where in vitro fertilization is used, the fertilized embryo may be implanted into a surrogate host or incubated in vitro, or both. Using either method, the progeny may be evaluated for the presence of the transgene using methods described above, or other appropriate methods.

The transgenic animals produced in accordance with the present invention will include exogenous genetic material. As set out above, the exogenous genetic material will, in certain embodiments, be a DNA sequence which results in the production of an Fc receptor. Further, in such embodiments the sequence will be attached to a transcriptional control element, e.g., a promoter, which preferably allows the expression of the transgene product in a specific type of cell.

Retroviral infection can also be used to introduce transgene into a non-human animal. The developing non-human embryo can be cultured in vitro to the blastocyst stage. During this time, the blastomeres can be targets for retroviral infection (Jaenich, 1986). Efficient infection of the blastomeres is obtained by enzymatic treatment to remove the zona pellucida (Manipulating the Mouse Embryo, Hogan et al. eds., 1986). The viral vector system used to introduce the transgene is typically a replication-defective retrovirus carrying the transgene (Jahner et al., 1985; Van der Putten et al., 1985). Transfection is easily and efficiently obtained by culturing the blastomeres on a monolayer of virus-producing cells (Van der Putten, 1985; Stewart et al., 1987). Alternatively, infection can be performed at a later stage. Virus or virus-producing cells can be injected into the blastocoele (Jahner et al., 1982). Most of the founders will be mosaic for the transgene since incorporation occurs only in a subset of the cells which formed the transgenic non-human animal. Further, the founder may contain various retroviral insertions of the transgene at different positions in the genome which generally will segregate in the offspring. In addition, it is also possible to introduce transgenes into the germ line by intrauterine retroviral infection of the midgestation embryo (Jahner et al. 1982).

A third type of target cell for transgene introduction is the embryonal stem cell (ES). ES cells are obtained from pre-implantation embryos cultured in vitro and fused with embryos (Evans et al., 1981; Bradley et al., 1984; Gossler et al., 1986; Robertson et al., 1986). Transgenes can be efficiently introduced into the ES cells by DNA transfection or by retrovirus-mediated transduction. Such transformed ES cells can thereafter be combined with blastocysts from a non-human animal. The ES cells thereafter colonize the embryo and contribute to the germ line of the resulting chimeric animal. For review see Jaenisch (1988).

### VIII. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Materials and Methods

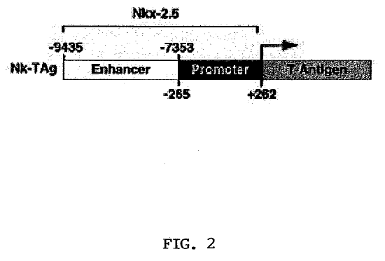

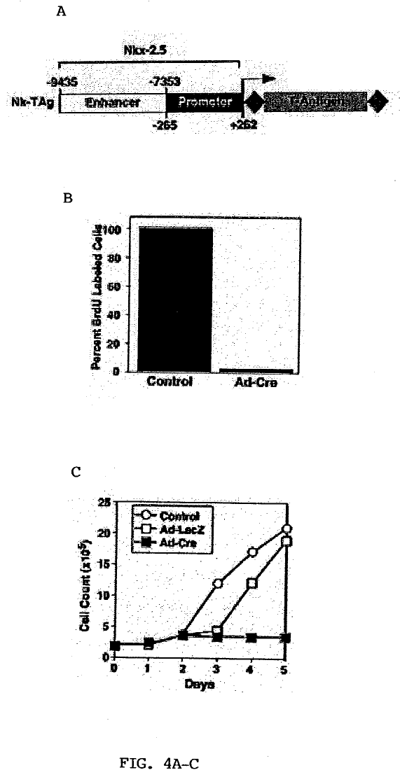

Generation of Transgenic Mice. DNA constructs, Nk-TAg (FIG. 2) and NkL-TAg (FIG. 4A), were used to generate transgenic mice expressing SV40 large TAg under the control of the mouse Nkx2.5 gene heart-specific enhancer (−9435 through−7353 bp) and its proximal promoter (−265 through +262 bp) (Lien et al., 1999). NkL-TAg DNA was constructed by inserting 34-bp loxP sequences on either side of the TAg of Nk-TAg DNA. The orientation of the loxP recognition sequences was confirmed by sequencing. Nk-TAg and NkL-TAg DNA fragments were excised from the pBluescript plasmid using XhoI and XbaI and gel-purified prior to microinjection of the DNA into pronuclei of fertilized B6C3F1 oocytes. Genotyping of F0 mice was performed by Southern blot and PCR analysis using genomic DNA. The probe used for Southern blot analysis was a 1209 bp BamHI fragment excised from the Nk-TAg construct. PCR genotyping was performed using primers for TAg, generating a 500 bp band. Primer sequences are as follows:

| | |
|---|---|
| TAg forward: | 5′-CGCCAGTATCAACAGCCTGTTTGGC-3′ |
| TAg reverse: | 5′CATATCGTCACGTCGAAAAAGGCGC-3′ |

Cell Culture. Cells were isolated from sub-endocardial tumor-like structures in the hearts of transgenic mice as previously described with some modifications (Paradis et al, 1996). Briefly, dissected tissues were minced and dissociated using an enzyme mix containing 0.2% collagen type II (Worthington) and 0.6 mg/ml of pancreatin (Sigma). Cardiac cells derived from Nk-TAg and NkL-TAg mice were maintained in DMEM/F12 media supplemented with 100 IU/ml penicillin, 100 μg/ml streptomycin, 2 mM Lglutamine, and 10% fetal bovine serum (FBS) on plates coated with 12.5 mg/ml fibronectin and 0.1% 2 gelatin. Cloning cylinders were used to harvest cell clones. Cell growth curves were generated by plating 1.5×10⁵ or 2×10⁵ cells (as indicated) in growth medium onto 100 mm plates and counting total cell number every 24 hours for 5 days. Differentiation medium contains DMEM/F12 supplemented with 100 IU/ml penicillin, 100 μg/ml streptomycin, 2 mM L-glutamine, 5% heat inactivated horse serum, 10 mg/ml insulin, 5.5 mg/ml transferrin and 6.7 ng/ml sodium selenite.

Histochemical Analysis. Heart tissue was fixed in 10% phosphate-buffered formalin and stained with hematoxylin-eosin. Nk-TAg and NkL-TAg cells were cultured on coverslips and fixed for 10 minutes with either −20° C. methanol or 4% paraformaldehyde for immunohistochemistry. Blocking was performed by incubating fixed cells with 1.5% bovine serum albumin and 10% normal goat serum in PBS for 20 minutes. Primary antibodies were incubated for 30-60 minutes in 1.5% bovine serum albumin in PBS as follows: monoclonal anti-myosin (smooth) (1:100, Sigma), polyclonal antimyosin (skeletal) (1:100, Sigma), monoclonal anti-α-smooth muscle actin (1:100, Sigma), monoclonal anti-skeletal myosin (slow) (1:100, Sigma), polyclonal anti-connexin 43 (1:100, Sigma), monoclonal anti-α-sarcomeric actin clone 5C5 (1:100, Sigma), monoclonal anti-desmin (1:100, Sigma), monoclonal anti-calponin (1:100, Sigma), monoclonal anti-α-actinin (sarcomeric) clone EA-53 (1:200, Sigma), monoclonal anti-SV40 T antigen (1:100, Santa Cruz). Secondary antibodies conjugated to either FITC or Texas Red (1:200, Vector Labs) were diluted in PBS and incubated for 30 minutes at room temperature. In some cases, nuclei were co-stained with DAPI (10 mg/ml) for 1 minute. Coverslips were mounted with Vectashield (Vector Laboratories) and fluorescence or confocal images were captured using Leica DMRXE or Zeiss 3.95 microscopes, respectively.

Measurements of Contractility and Calcium Transients in Response to E1 ectrical Stimulation. Excitation-contraction coupling function was assessed as previously described (Cyran et al., 1992; and De Young et al., 1989) by measuring cell contractility and calcium transients. Briefly, cells were grown in 35-mm tissue culture dishes for 2-3 days until 70-80% confluent and loaded with 2 mM fura 2-AM (Molecular Probes) in MEM containing 0.5 mM probenecid for 15 minutes at 37° C. A platinum electrode ring was placed in the tissue culture dish. Myocyte contractions and calcium transients were elicited by field stimulation at 1.5 Hz (Ion Optix) with current pulses of 4 msec duration and voltages of 40V. The polarity of the stimulating electrodes was alternated at every pulse to prevent accumulation of electrochemical byproducts. Myocyte contractions were imaged and scanned at a rate of 240 HZ (Ion Optix). Calcium transients were observed by exciting the fura-2 AM loaded cells with alternating wavelengths of 340 and 380 nm, and recording the emission intensity at 510 nm. Contraction and calcium transient data for each myocyte were recorded from a minimum of 12 consecutive stimuli.

Drug treatment. Drugs were added into cell culture media as indicated at the following concentrations: 100 mM phenylephrine (PE), 10 mM norepinephrine (NE), 10 ng/ml recombinant human transforming growth factor-β1 (TGFβ1) (R&D Systems), 1 mM dynorphin-β (Peninsula Laboratories), 1 mM trans- or cis-retinoic acid (Sigma), 15 ng/ml bone morphogenetic protein (BMP)-2/4 (Genetics Institute, Cambridge, Mass.), 1 mM angiotensin II (R&D Systems), 20 nM endothelin-1 (ET-1) (R&D Systems), 100 ng/ml insulin-like growth factor I (IGF-I) (Roche), 5-azacytidine (Sigma), and 10 mg/ml mitomycin C (Sigma).

Viral Infection. Cells were infected with recombinant adenoviruses (Ad) at a multiplicity of infection (MOI) of 100 for 3 to 12 hrs. The medium was replaced with growth medium, and NkL-TAg cells were cultured for the indicated times before analyses. Recombinant adenoviruses were obtained from the following sources: Ad-Cre recombinase (Ad-Cre) was provided by Dr. Frank Graham (McMaster University) (Anton and Graham, 1995); GATA4 (Ad-GATA4), Nkx2.5 (Ad-Nkx2.5), MEK6 (Ad-MEK6), and GFP (Ad-GFP) were generated using the “Easy-Track” system as described (He et al., 1998); antisense HDAC4 and HDAC5 (Ad-HDAC4 or 5), expressing coding regions of HDAC genes in reverse orientation, were provided by Chun Zhang; MEF2C (Ad-MEF2C) was provided by Rebekka Nicol (HDAC 4/5 and MEF2c viruses were generated using pAC-CMV vector); constitutively active calcineurin (Ad-CnA), IGF-I receptor (Ad-IGFI), constitutively active CaMKI (Ad-CaMKI), β-galactosidase (Ad-LacZ) were generated by Dr. Robert Gerard (UT Southwestern) and were constructed using an in vitro Cre-lox recombination system (Aoki et al., 1999; Ng et al. 1999).

DNA Synthesis Assay. NkL-TAg cells were infected with Ad-Cre virus as described above and cultured in growth medium for two more days followed by incubation with BrdU for 2 hours. DNA synthesis was determined based on BrdU incorporation using a BrdU assay kit (Roche) according to manufacturer's instructions.

Western Blot Analysis. Cells were lysed in 10 mM Tris-HCl (pH 6.8), 100 mM NaCl, 1% SDS, 1 mM EDTA, 1% EGTA buffer. Total protein (50 mg) from lysate was resolved on a 10% SDS-PAGE gel and transferred to a PVDF membrane. Membranes were incubated with primary antibodies recognizing GATA4 (Santa Cruz), and TAg (Santa Cruz) at 1:1000 dilution followed by incubation with secondary antibodies against rabbit IgG or mouse IgG (1:7500) conjugated to HRP (Santa Cruz). Signal was detected using ECL reagent (Amersham).

RT-PCR and Northern Blot Analysis. RNA was isolated from cells using Trizol Reagent (Invitrogen) and used in Northern blot analysis with a probe generated from the coding region of Nkx2.5 or TAg. RT-PCR was performed using the Superscript II kit (Invitrogen). Primers used for amplification are listed in the Table 2.

Microarray Hybridizations. Microarray analyses for NkL-TAg cells versus NIH3T3 cells were performed using the InCyte Genomics Mouse cDNA microchip. Microarray analyses for NkL-Tag cells infected with Ad-Cre or Ad-lacZ were performed at the UT Southwestern Core Facility using Affymetrix mouse oligo microchip (MU74A). NkL-TAg cells were infected either with adenovirus expressing Cre recombinase (Ad-Cre) or β-galactosidase (Ad-LacZ) at 100 MOI per cell for three hours. Cells were incubated for 4 days in differentiation media (described above) containing 100 mM PE followed by RNA isolation using Trizol reagent (Invitrogen). Data were analyzed using GeneSpring and Affymetrix Suite software.

### Example 2

### Results

Generation of Nkx2.5-TAg transgenic mouse with cardiac tumor. The Nkx2.5 cis-regulatory sequences consisting of the early cardiac-specific enhancer region (−9435 to −7353 bp) fused to the endogenous promoter (−265 to +262 bp) was linked to the coding region for SV-40 large T-antigen (FIG. 2) and used to generate transgenic mice. Founder transgenic mice appeared normal at birth and showed no abnormalities during the neonatal period. However, one female (F₀) transgenic mouse died spontaneously at 5 weeks of age. An autopsy showed that the heart was grossly enlarged with multiple sessile masses protruding into the left ventricular chamber from the interventricular septum and anterior surface of the ventricular free wall. Histological analysis confirmed that the masses were localized subendocardially and consisted of small poorly differentiated, spindle shaped cells with small cosin-rich cytoplasm without apparent striation. Many loci of myocardial hyperplasia were noted, none of which involved the endocardium. The architecture of the remaining myocardium was preserved although many cardiomyocytes had excessively large hemotoxylin-rich nuclei as a possible sign of polyploidy. It was not possible to determine the cause of death of the animal; however, it is plausible that either outflow obstruction or ventricular arrhythmia led to sudden death.

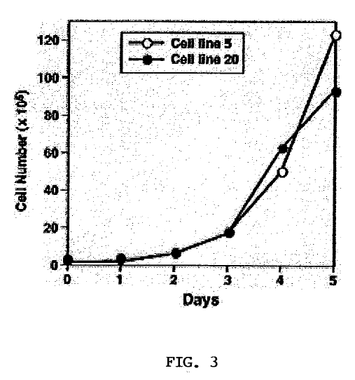

Isolation of immortalized cardiac cells. A heart harvested from a 3.5 week-old (F₀) transgenic Nkx2.5-TAg mouse displaying mild cyanosis exhibited protruding masses in the left ventricle. These tumors were excised from the myocardium and dissociated into single cells. The cells were seeded at low density onto fibronectin/gelatin-coated plates and grown for 10 days. During this period of time several well-defined cell colonies emerged consisting of small spindle-shaped cells growing on top of each other. They showed no contractile activity. Cell colonies were cloned using cloning cylinders and independently sub-cultured in 24-well plates. Twenty-one individual colonies were isolated, although only eighteen survived subsequent passages. These cell lines were named Nk-Tag lines. Following dissociation of the colonies, the plated cells grew as a monolayer. Different growth rates were observed for individual clones (FIG. 3). Upon reaching confluence the cells continued to proliferate showing no evidence of contact inhibition. When cells were plated at 1×106 cells per 10 cm plate and grown in medium containing 20% FBS, we observed a doubling time of less than 24 hours. Decreasing the serum content to 15% slightly reduced the doubling time indicating that Nk-TAg cells respond to factors in serum.

Gene expression profile of Nk-TAg cells. All of the Nk-TAg cell lines expressed TAg, but expression levels of TAg varied in the different cell lines. A correlation was seen between the expression level of TAg and growth rate of cells. However, there was no relation seen between expression levels of TAg and cardiac-enriched gene expression. The majority of Nk-TAg cell lines expressed transcripts encoding proteins characteristic of cardiomyocytes, such as the Nkx2.5, GATA-4, and MEF2C transcription factors (see Table 1 for a list of other genes). However, many structural proteins that are essential for contraction of cardiac myocytes, including titin and sodium, calcium-exchanger (Ncx-1), were not detected even by RT-PCR (Table 1). Immunohistochemistry analysis of Nk-TAg cells showed a low level of expression of α-actinin, a prototypical Z-line protein in cardiomyocytes. Growing the cells on plates coated with laminin or type II collagen at normal (10%) or low serum content (2 or 5%) did not increase expression of α-actinin. However, addition of mitomycin C to the growth medium abated proliferation of the Nk-TAg cells and enhanced expression of α-actinin in the cytoplasm as unassembled Z-lines. This finding suggested that inhibition of DNA synthesis in Nk-TAg cells promoted a cardiogenic phenotype. Addition of hypertrophic agents including ET-1, PE, and angiotensin II did not further induce the assembly of sarcomeres in Nk-TAg cells.

Generation of conditional TAg-transformed cardiomyocyte cell line. Based on the finding that mitomycin C enhanced α-actinin expression in Nk-TAg cells, inventors postulated that the lack of growth control is counterproductive to establishing a cardiomyocyte cell line. Therefore, in an effort to induce cellular differentiation the inventors sought to generate cardiac cell lines capable of terminating cell division. Inventors chose the Cre-lox system as an efficient method to permanently remove and thereby inactivate TAg. Two loxP sites were added to flank the TAg encoding region in the Nk-TAg DNA construct (FIG. 4A) and this construct, NkL-TAg, was used to generate transgenic mice. Dissection of the hearts of NkL-TAg transgenic mice at 3.5 weeks of age revealed one mouse with gross cardiomegaly due to multiple ventricular myocardial tumors similar to those found in Nk-TAg mice. Histological analysis of the transgenic heart revealed a phenotype similar to that observed in the hearts of Nk-TAg transgenic animals. Cells were dissociated from the tumor regions of the NkL-TAg transgenic hearts and plated onto fibronectin/gelatin-coated dishes at a low density. Following 10 days in growth medium containing 10% FBS, the NkL-TAg cells showed different characteristics than the Nk-TAg cells. In contrast to the Nk-TAg cells, NkL-TAg cells did not form well-defined colonies and did not grow on top of each other. Remarkably, some of the plated cells maintained contractile activity during the initial passages, although this characteristic gradually disappeared after a few cell passages. Immunohistochemistry confirmed TAg expression in the NkL-TAg cells and also showed that some cells expressed α-actinin in a non-striated pattern.

NkL-TAg cells exit the cell cycle following Cre-recombinase expression. NkL-TAg cells grew without apparent senescence even after 50 serial cell passages. Immunocytochemistry using TAg antibody showed that all NkL-TAg cells expressed TAg protein regardless of the passage number. Infection of NkL-TAg cells with recombinant adenovirus expressing Cre recombinase (Ad-Cre) effectively removed the TAg gene from the cellular genome as confirmed by PCR and immunocytochemistry using TAg antibody. Deletion of the TAg gene was followed by a dramatic decrease in BrdU incorporation (FIG. 4B) and eventual cessation of cell growth of the NkL-TAg cells (FIG. 4C). In contrast, infection of NkL-TAg cells with a recombinant adenovirus expressing β-galactosidase (Ad-LacZ) initially caused a decrease in the cell growth rate, but ultimately resulted in no change in cell proliferation rate when compared to non-infected cells (FIG. 4C).

Characterization of NkL-TAg cells before and after removal of Tag. Although NkL-TAg cells showed an expression pattern similar to Nk-TAg cells (Table 1), additional cardiac-specific genes were detected in NkL-TAg cells, including: myoglobin, α-myosin heavy chain (α-MHC), FHL2/DRAL, MCIP1, MEF2D and ANF. Immunocytochemistry revealed expression of desmin and cadherin as well as connexin43 that was localized to the cell junctions. Deletion of the TAg and subsequent withdrawal from the cell cycle resulted in a dramatic increase in cell surface area (up to 10-fold and dependent on cell density). Immunocytochemistry showed that NkL-TAg cells depleted of TAg exhibited pronounced stress fibers that were immunoreactive for smooth muscle actin. After removal of TAg, many of the NkLTAg cells become binucleated. However, depletion of TAg had no apparent effect on the level of expression of selected cardiac-specific genes as tested by RT-PCR and/or immunocytochemistry.

Induction of differentiation of NkL-TAg cells by various stimuli. Inventors surveyed the effect of different drugs, hormones and overexpression of signaling proteins in attempts to induce further differentiation of NkL-TAg cells. Differentiation was assessed using immunocytochemisrty with an antibody against sarcomeric α-actinin. Prior to depletion of TAg, addition of caffeine, potassium chloride, 5-azacytidine, low oxygen, PE, NE, IGF-I, cis- or trans-retinoic acids, dynorphin, TGFb1, ionomycin, basic fibroblast growth factor (bFGF) or recombinant adenovirus containing various transcripts (CMV-LacZ, CMV-Calsarcin1, CMV-GATA4, CMV-Nkx2.5, CMV-IGFI, CMV-asHDAC5, CMV-CaMKI, CMV-MEK6, CMV-Calcineurin A) had no effect on inducing sarcomere formation as assessed by α-actinin immunocytochemistry. However, expression of α-actinin was increased in NkL-TAg cells upon removal of TAg and switching the medium to contain low serum (5% horse serum), insulin, transferrin, and selenium. Moreover, addition of PE, BMP2/4, or NE, to the media led to further induction of sarcomere formation in some cells. A similar induction was seen when NkL-TAg cells depleted of TAg were grown in differentiation media and infected with recombinant adenovirus expressing constitutively active CaMKIV or constitutively active calcineurin A, known effectors of cardiomyocyte hypertrophy (Passier et al., 2000; Molkentin et al., 1998).

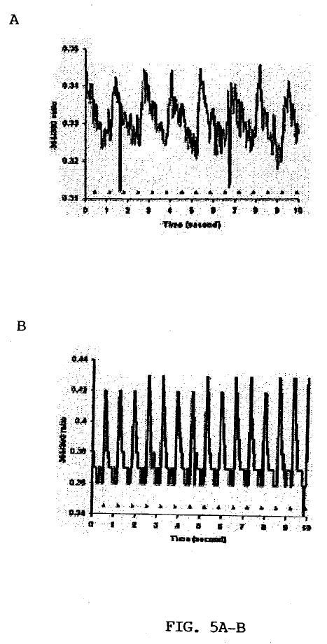

NkL-TAg cells exhibit calcium transients in response to electrical stimulation. Calcium current and cell contractility were analyzed to determine whether NkL-TAg cells were excitable. NkL-TAg cells expressing TAg showed no response to electrical stimulation. However, when NkL-TAg cells depleted of TAg and cultured for seven days in differentiation medium containing PE, were subjected to electrical stimulation, calcium transients were readily detected in approximately 30% of the cells examined (FIG. 5A). This implies that the sarcoplasmic reticulum of NkL-TAg cells releases calcium in response to electrical stimulation. Despite this fact, cells failed to contract. In comparison to freshly isolated adult cardiac myocytes (FIG. 5B), NkL-TAg cells showed a diminished response. In addition, calcium transients in NkL-TAg cells were elicited in response to every other stimulus at the 50 Hz stimulation frequency, whereas freshly isolated cardiac myocytes responded to each electrical stimulus. The inability of NkL-TAg cells to respond to every stimulus may be attributable to delayed restoration of excitability since intracellular calcium in these cells may return to the basal level more slowly than in normal adult myocytes.

Microarray analysis of Nk-TAg and NkL-TAg cell lines. Gene expression profiles were examined using microarray analysis comparing RNA transcripts of NkL-TAg cells with NIH/3T3 fibroblasts. Scatter plot analysis of the microarray results revealed three distinct groups of genes. The first and largest group contains genes that are equally represented in both of the cell lines, consisting primarily of housekeeping genes. The second group contains genes that are predominantly or exclusively expressed in NIH/3T3 cells and consists of non-muscle genes. The third group of genes is predominantly or exclusively expressed in NkL-TAg cells and consists mainly of cardiac-specific genes, supporting the premise that the NkL-TAg cell line is a cardiomyocyte cell line (Table 3). Notably, many of the genes revealed by microarray analysis, such as those encoding calponin, smooth muscle actin, skeletal actin, and ANF are characteristic of embryonic cardiomyocytes. Furthermore, analysis of the microarray data revealed several uncharacterized ESTs expressed in NkL-TAg cells. These ESTs were shown to be selectively expressed in heart by Northern blot and in situ hybridization. Microarray analysis was also performed on RNA transcripts isolated from NkL-Tag cells (grown in differentiation media with PE) expressing or depleted of TAg. Infection of NkLTAg cells with Ad-LacZ showed an up-regulation of 298 genes, 197 of these genes were also upregulated in NkL-TAg cells infected with Ad-Cre, and 232 genes were down-regulated, including 74 genes that were down-regulated in Ad-Cre infected cells. Further studies were not done on these genes, viewing them as a cellular response to adenovirus infection. Deletion of the TAg gene by Cre-recombinase led to significant alterations of gene expression in NkL-TAg cells, 313 genes were down regulated (>2-fold) and 214 genes were up-regulated (>2-fold). Specifically, the majority of genes down-regulated in TAg-deleted NkL-TAg cells were involved in cell cycle regulation, including those encoding: cyclin A2, cyclin E2, cyclin B1, cyclin B2, cdc45, cdc46, cdc6, cdc7, Mcm2, cdc25 and cdk2. In contrast, many of the transcripts that were up-regulated upon deletion of TAg were cardiac-associated genes including those encoding: slow/cardiac troponin C; ZASP, a cardiac-specific Z-band protein; skeletal muscle actin, brain natriuretic peptide, fatty acid transport protein 4, myotonic dystrophy protein kinase and sarcoglycan, a dystrophin-associated glycoprotein. Expression of some of these genes was confirmed with immunohistochemistry on NkL-TAg cells in the absence or presence of Ad-Cre.

TABLE 1
Genes Expressed in Nk-TAg

| GENE | Line 5 | Line 20 |
|---|---|---|
| T-antigen | + | + |
| Nkx-2.5 | + | + |
| Gata4 | + | + |
| Myocardin | + | + |
| MEF2c | + | |
| Oracle | + | + |
| CHAMP | + | + |
| Tropomyosin | + | + |
| beta-MHC | + | + |
| Alpha-MHC | − | + |
| Troponin I | + | + |
| alpha-Cardiac Actin | + | + |
| Connexin 45 | + | + |
| Dystrophin | − | − |
| Myoglobin | − | − |
| MURF2 | − | − |
| Calsarcin | − | + |
| MLC2v | − | + |
| KvLQT | + | + |
| Titin | − | − |
| ANF | − | − |
| Ncx-1 | − | − |

TABLE 2
Primer Sequences

| | |
|---|---|
| β-MHC | TGC AAA GGC TCC AGG TCT GAG GGC |
| (f) | |
| β-MHC | GCC AAC ACC AAC CTG TCC AAG TTC |
| (r) | |
| α-MHC | CTG CTG GAG AGG TTA TTC CTC G |
| (f) | |
| α-MHC | GGA AGA GTG AGC GGC GCA TCA AGG |
| (r) | |
| ANF (f) | ACC TGC TAG ACC ACC TGG AGG AG |
| ANF (r) | CCT TGG CTG TTA TCT TCG GTA CCG G |
| BNP (f) | ATC TCC TGC AGG TGC TGT CCC AG |
| BNP (r) | GGT CTT CCT ACA ACA ACT TCA GTG CGT TAC |
| MLC2v | CAG ATC CAG GAG TTC AAG GAA GCC TT |
| (f) | |
| MLC2v | CTT TGG AGA ACC TCT CTG CTT GTG TGG |
| (r) | |
| TnI | TGC CGG AAG TTG AGA GGA AAT CCA AGA T |
| (slow) | |
| (f) | |
| TnI | CCA GCA CCT TCA GCT TCA GGT CCT TGA T |
| (slow) | |
| (r) | |
| Dystro- | CAT TCA AGA AGT GGA AAT GTT GCC CAG G |
| phin | |
| (f) | |
| Dystro- | CTC GGC AGA AAG AAG CCA TGA AAG TAC |
| phin | |
| (r) | |
| Titin | GGA CCA AAC CTA TCT ATG ATG GTG GC |
| (f) | |
| Titin | GGA ACA AAC AGC CTT AAG GAA CCA C |
| (r) | |
| Tropo- | AAG ATG CAG ATG CTG AAG CTC GAC |
| myosin | |
| (f) | |
| Tropo- | CTC CAG CTT CTG CAG AGC TGT G |
| myosin | |
| (r) | |
| GAPD (f) | GCA GTG GCA AAG TGG AGA TTG |
| GAPD (r) | TTT GGC TCC ACC CTT CAA GTG |
| GATA4 | TCA ATT GTG GGG CCA TGT CCA |
| (f) | |
| GATA4 | TGA ATC CCC TCC TTC CGC ATT |
| (r) | |
| Kcnel | CTA GAC CCA GGA GTT TTG CTC |
| (f) | |
| Kcnel | CTC TGA AGC TCT CCA GGA CAC |
| (r) | |
| KVLQT1 | GAT AGG AGG CCA GAC CAT TTC |
| (f) | |
| KVLQT1 | CTG ATC CAG CCT TCT CTG TAG |
| (r) | |
| Mef2c | GGA TCC TTG GGA GAA AAA AGA TTC AGA TTA C |
| (f) | |
| Mef2c | GTC TAG ACT ACC CAC CGT ACT CGT CAA T |
| (r) | |
| Merg 1b | GGC CCA GGA GGT CCT GTC C |
| (f) | |
| Merg 1b | GTG GCC CAG GAG GTC CTG TC |
| (r) | |
| Ncx 1 | TCC TCG TCA TCG ATT ACC TTG A |
| (f) | |
| Ncx 1 | GAG AGC ATT GGC ATC ATG GAG |
| (r) | |

TABLE 3
Selected Genes Preferentially Expressed in the NkL-TAg Cells
Compared to the NIH/3T3 Cells

| | | Fold |
|---|---|---|
| NCBI # | Gene Name | Diff. |
| AA671284 | troponin T2, cardiac | 41.1 |
| AI326574 | Skeletal muscle LIM-protein 1 | 20.8 |
| | (4 ½ LTM domains 1) | |
| AA880322 | calponin 1 | 19.6 |
| AA792499 | CARP (ankyrin-like repeat protein) | 19.5 |
| AA879966 | laminin, alpha 5 | 12.9 |
| AA624460 | actin, alpha 2, smooth muscle, aorta | 11.7 |
| AA674109 | sarcoglycan, epsilon | 7.1 |
| AA606940 | ADP-ribosylation-like factor 6 interacting protein | 6.7 |
| AA795463 | myomesin 1 (titin-associated protein) | 6.3 |
| AI604642 | insulin-like growth factor 1 | 5.8 |
| AA619890 | TGF b2 (transforming growth factor, beta 2) | 5.4 |
| W18330 | tropomyosin 2, beta | 5.1 |
| AA522219 | phospholipase C, beta 3 | 5 |
| AA799087 | mitogen activated protein kinase kinase kinase 1 | 5 |
| AA756136 | actin, gamma 2, smooth muscle, enteric | 4.8 |
| AA288642 | cholinergic receptor, nicotinic, beta | 4.2 |
| | polypeptide 1 (muscle) | |
| AA770902 | actin, alpha 1, skeletal muscle | 4.1 |
| AA261149 | procollagen, type XVIII, alpha 1 | 4 |
| AA671340 | selenoprotein R | 3.9 |
| AI325745 | actin, alpha, cardiac | 3.9 |
| AA681115 | G protein-coupled receptor kinase 5 | 3.8 |
| AA403815 | desmoglein 2 | 3.7 |
| AI604588 | enigma homolog 1 (heart/skeletal muscle-specific) | 3.7 |
| W15812 | desmin | 3.5 |
| AA738914 | gap junction membrane channel protein alpha 1 | 3.4 |
| AA718467 | S100 calcium binding protein Al (heart) | 3.4 |
| AA547343 | integrin alpha V (Cd51) | 3.4 |
| AI036489 | cyclin G | 3.4 |
| AA123128 | integrin beta 1 (fibronectin receptor beta) | 3.4 |
| AA815681 | CLP-36 (Elfin; Heart & Skeletal Muscle; | 3.3 |
| | Interacts w/Actinin) | |
| AI882290 | Rho-associated coiled-coil forming kinase | 3.2 |
| | 2 (ROCK 2) | |
| AI391322 | protein kinase inhibitor, alpha | 3.2 |
| AA003303 | tight junction protein 2 | 2.9 |
| AA881654 | vinculin | 2.8 |
| AA879643 | insulin-like growth factor binding protein 2 | 2.7 |
| AA718314 | FXYD domain-containing ion transport regulator 5 | 2.7 |
| AI226235 | gap junction membrane channel protein beta 4 | 2.7 |
| AA617613 | ras-GTPase-activating protein (GAP120) | 2.6 |
| AA674780 | cyclin-dependent kinase inhibitor 2D | 2.6 |
| | (p19, inhibits CDK4) | |
| AW210300 | chloride channel 3 | 2.6 |
| AW210329 | tight junction protein 1 | 2.5 |
| AI894082 | aortic preferentially expressed gene 1 | 2.5 |
| AA066778 | Ras suppressor protein 1 | 2.5 |
| AA166386 | transducer of ErbB-2.1 | 2.5 |
| AI048103 | Rab6, kinesin-like | 2.5 |
| W34124 | calponin 2 | 2.5 |
| AA536899 | GATA-binding protein 6 | 2.5 |
| AA792278 | endothelin 1 | 2.5 |
| AA624474 | integrin linked kinase | 2.5 |
| AA880220 | jagged 1 | 2.5 |
| AA645955 | nidogen 2 | 2.5 |
| AIS94824 | endoglin | 2.5 |
| W76764 | regulator of G protein signaling 7 | 2.4 |
| AA434955 | catenin alpha 1 | 2.4 |
| W47897 | large tumor suppressor 2 | 2.4 |
| AA553029 | cadherin 5 | 2.3 |
| AA067836 | cyclin-dependent kinase inhibitor | 2.3 |
| | 2B (p15, inhibits CDK4) | |
| AA879568 | cyclin D2 | 2.2 |
| AA437625 | geminin | 2.1 |
| AA073952 | junction plakoglobin | 2.1 |
| AA066685 | villin 2 | 2.1 |
| AA437512 | FK506 binding protein 6(65 kDa) | 2.1 |
| AA242226 | cadherin 2 | 2.1 |
| AA684114 | nibrin | 2 |
| AA606686 | myosin light chain, alkali, nonmuscie | 2 |
| AA666992 | GATA-binding protein 3 | 2 |
| AA467584 | cadherin 3 | 2 |
| AI894359 | coronin, actin binding protein 1C | 2 |
| AA437878 | Rho-associated coiled-coil forming kinase 1 | 1.9 |
| | (ROCK 1) | |
| AA623765 | procollagen, type V, alpha 2 | 1.9 |
| AA221467 | cadherin 4 | 1.9 |
| AA386846 | calsequestrin 2 | 1.9 |
| AA815689 | troponin C, cardiac/slow skeletal | 1.9 |
| AA435278 | calsequestrin 1 | 1.8 |
| AA072780 | retinoblastoma 1 | 1.8 |
| AI197413 | heart & neural crest derivatives expressed | 1.7 |
| | transcript 1 (eHAND) | |
| AA003458 | ATPase, Ca++ transporting, cardiac | 1.7 |
| | muscle, slow twitch 2 | |
| AA413490 | transferrin receptor | 1.7 |
| AA718365 | bone morphogenetic protein 6 | 1.6 |
| AA755870 | natriuretic peptide precursor type A | 1.6 |

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.

U.S. Pat. No. 4,684,611

U.S. Pat. No. 4,797,368

U.S. Pat. No. 4,952,500

U.S. Pat. No. 4,959,317

U.S. Pat. No. 5,139,941

U.S. Pat. No. 5,302,523

U.S. Pat. No. 5,322,783

U.S. Pat. No. 5,384,253

U.S. Pat. No. 5,464,765

U.S. Pat. No. 5,538,877

U.S. Pat. No. 5,538,880

U.S. Pat. No. 5,550,318

U.S. Pat. No. 5,563,055

U.S. Pat. No. 5,580,859

U.S. Pat. No. 5,589,466

U.S. Pat. No. 5,610,042

U.S. Pat. No. 5,656,610

U.S. Pat. No. 5,670,488

U.S. Pat. No. 5,702,932

U.S. Pat. No. 5,736,524

U.S. Pat. No. 5,780,448

U.S. Pat. No. 5,789,215

U.S. Pat. No. 5,945,100

U.S. Pat. No. 5,981,274

U.S. Pat. No. 5,994,624

Abremski et al., Cell 32:1301-1311, 1983.

Anton and Graham, J. Virol. 4600-4606, v.69(8), 1995.

Aoki et al., Mol. Med., 5 (4):224-31, 1999.

Baichwal and Sugden, In: Gene Transfer, Kucherlapati (ed.), New York, Plenum Press, 117-148, 1986.

Barnes et al., J. Biol. Chem., 272(17):11510-11517, 1997.

Bhavsa et al., Genomics, 35(1): 11-23, 1996.

Bradley et al., Nature 309:255-258, 1984.

Brandt et al., J. Cell Physiol., 88(3):255-275, 1976.

Brunskill et al., Dev. Biol. 235, 507-520, 2001.

Chang et al., Hepatology, 14:134A, 1991.

Chen and Okayama, Mol. Cell Biol., 7(8):2745-2752, 1987.

Clark et al., Hum. Gene Ther., 6(10):1329-1341, 1995.

Coffin, Retroviridae and Their Replication. In: Virology, Fields et al., eds., Raven Press, New York, pp. 1437-1500, 1990.

Couch et al., Am. Rev. Resp. Dis., 88:394-403, 1963.

Couparetal., Gene, 68:1-10, 1988.

Cyran et al., J. Mol. Cell. Cardiol, 241:1167-1177, 1992.

De Young et al., Am. J. Physiol., 257:C750-C758, 1989.

Engelmann et al., J. Mol. Cell. Cardiol. 25, 197-213, 1993.

EPO 0 273 085

Evans et al., (1981) Nature 292:154-156

Fechheimer et al., Proc. Nat'l Acad. Sci. USA, 84:8463-8467, 1987.

Flotte et al., Am. J. Respir. Cell. Mol. Biol., 7(3):349-356, 1992.

Flotte et al., Gene Ther., 2(1):29-37, 1995.

Flotte et al., Proc. Nat'l Acad. Sci. USA, 90(22):10613-7, 1993.

Fraley et al., Proc. Nat'l Acad. Sci. USA, 76:3348-3352, 1979.

Franz W M, et al., Cardioscience, 5(4):235-43, 1994.

Friedmann, Science, 244:1275-1281, 1989.

Ghosh and Bachhawat, In: Liver Diseases, Targeted Diagnosis and Therapy Using Specific Receptors and Ligands. Wu et al., eds., Marcel Dekker, New York, pp. 87-104, 1991.

Ghosh-Choudhury et al., EMBO J., 6:1733-1739, 1987.

Gomez-Foix et al., J. Biol. Chem., 267:25129-25134, 1992.

Gopal, Mol. Cell Biol., 5:1188-1190, 1985.

Gopal-Srivastava et al., J. Mol. Cell. Biol. 15(12):7081-90, 1995.

Gossler et al., Proc. Nat'l Acad. Sci. USA 83: 9065-9069, 1986.

Graham and Prevec, Biotechnology, 20:363-390, 1992.

Graham and Prevec, In: Methods in Molecular Biology: Gene Transfer and Expression Protocol, Murray (Ed.), Humana Press, Clifton, N.J., 7:109-128, 1991.

Graham and van der Eb, Virology, 52:456-467, 1973.

Graham et al., J. Gen. Virol., 36:59-72, 1977.

Grunhaus et al., Seminar in Virology, 200(2):535-546, 1992.

Gu et al., Cell 72, 309-324, February 1993.

Harland and Weintraub, J. Cell Biol., 101:1094-1099, 1985.

He et al., Proc. Natl. Acad. Sci., 95:2509-2514, 1998.

Hermonat and Muzycska, Proc. Nat'l Acad. Sci. USA, 81:6466-6470, 1984.

Hersdorffer et al., DNA Cell Biol., 9:713-723, 1990.

Herz and Gerard, Proc. Nat'l Acad. Sci. USA, 90:2812-2816, 1993.

Hoess et al., Proc. Nat'l Acad. Sci. USA, 79:3398, 1982.

Horwich et al., Virol., 64:642-650, 1990.

Ito et al., Nuc. Acid Res. 10:1755. 1982.

Jaenich, Proc. Nat'l Acad. Sci. USA 73:1260-1264, 1986.

Jaenisch, Science 240:1468-1474, 1988.

Jahn et al., J. Cell Science, 109:397-405, 1996.

Jahner et al., Nature 298:623-628, 1982.

Jahner et al., Proc. Nat'l Acad. Sci. USA 82:6927-6931, 1985.

Jones and Shenk, Cell, 13:181-188, 1978.

Kaneda et al., Science, 243:375-378, 1989.

Kaplitt et al., Nat Genet., 8(2):148-54, 1994.

Karlsson et al., EMBO J., 5:2377-2385, 1986.

Kasahara et al., Science, 266(5189):1373-6, 1994.

Kato et al., J. Biol. Chem., 266:3361-3364, 1991.

Kelly et al., J. Cell. Biol., 129(2):383-96, 1995.

Kimes and Brandt, Exp. Cell Res., 98(2):367-381, 1976.

Kimura et al., Dev. Growth Differ. 39(3):257-65, 1997.

Klein et al., Nature, 327:70-73, 1987.

Kline et al., J. C. V. E1 ectrophys., 4(6), 642-660, 1993.

Klug et al., J. Clin. Invest. 98, 216-224, 1996.

Kotin et al., Proc. Nat'l Acad. Sci. USA, 1990 March;87(6):2211-5, 1990.

LaFace et al., Virology, 162(2):483-486, 1988.

LaPointe et al., Hypertension 27(3 Pt 2):715-22, 1996.

Laughlin et al., J. Virol., 60(2):515-524, 1986.

Le Gal La Salle et al., Science, 259:988-990, 1993.

Lebkowski et al., Mol. Cell. Biol., 8(10):3988-3996, 1988.

Levrero et al., Gene, 101:195-202, 1991.

Lien et al., Development, 126(1):75-84, 1999.

Manfredi & Prives, “The transforming activity of simian virus 40 large tumor antigen,” Biochim Biophys Acta 1198, 65-83, 1994.

Manipulating the Mouse Embryo, Hogan et al. eds., Cold Spring Harbor Laboratory Press, Cold Spring Harbor, 1986

Mann et al., Cell, 33:153-159, 1983.

Markowitz et al., J. Virol., 62:1120-1124, 1988.

McCarty et al., J. Virol., 65(6):2936-45, 1991.

McLaughlin et al., J. Virol., 62(6):1963-1973, 1988.

Miller et al., Am. J. Clin. Oncol., 15(3):216-221, 1992.

Miller et al., Mol. Cell. Biochem. 136, 29-34, 1994.

Molkentin et al., Cell, 93:215-228, 1998.

Morgan et al., Devel. Biol. 162, 486-498, 1994.

Moss et al., Biol. Chem., 271(49):31688-94, 1996.

Mouly et al., Exp. Cell Res. 225, 268-276, 1996.

Muzyczka, Curr. Topics Microbiol. Immunol., 158:97-129, 1992.

Ng et al., Human Gene Therapy, 10(16) 2667-72. 1999.

Nicolas and Rubinstein, In: Vectors: A survey of molecular cloning vectors and their uses, Rodriguez and Denhardt, eds., Stoneham: Butterworth, pp. 494-513, 1988.

Nicolau and Sene, Biochim. Biophys. Acta, 721:185-190, 1982.

Nicolau et al., Methods Enzymol., 149:157-176, 1987.

Ng et al., Hum. Gene Ther., 10:2667-2672, 1999.

Ogilvie et al., Science 214:270, 1981

Ohi et al., Gene, 89(2):279-282, 1990.

Omirulleh et al., Plant Mol. Biol., 21(3):415-28, 1993.

Paradis et al., J. Biol. Chem., 271(18):10827-33, 1996.

Parmjit, Proc. Nat'l Acad. Sci. USA 88, 5096-5100, 1991.

Passier et al., J. Clin. Invest., 105:1395-1406, 2000.

Paskind et al., Virology, 67:242-248, 1975.

PCT Application WO/93/19172

PCT Application WO 95/06128

PCT Application WO/94/09699

Perales et al., Proc. Nat'l Acad. Sci. USA, 91:4086-4090, 1994.

Potrykus et al., Mol. Gen. Genet., 199(2):169-77, 1985.

Potter et al., Proc. Nat'l Acad. Sci. USA, 81:7161-7165, 1984.

Racher et al., Biotechnology Techniques, 9:169-174, 1995.

Ragot et al., Nature, 361:647-650, 1993.

Renan, Radiother. Oncol., 19:197-218, 1990.

Rich et al., Hum. Gene Ther., 4:461-476, 1993.

Ridgeway, In: Vectors: A survey of molecular cloning vectors and their uses, Stoneham: Butterworth, pp. 467-492, 1988.

Rippe et al., Mol. Cell Biol., 10:689-695, 1990.

Ritchie, J. Biol. Chem. 271(41):25485-25491, 1996.

Robertson et al., Nature 322:445-448, 1986

Rosenfeld et al., Cell, 68:143-155, 1992.

Rosenfeld et al., Science, 252:431-434, 1991.

Roux et al., Proc. Nat'l Acad. Sci. USA, 86:9079-9083, 1989.

Samulski et al., EMBO J., 10:3941-3950, 1991.

Samulski et al., J. Virol, 63:3822-3828, 1989.

Sauer, Methods of Enzymology, 225, 890-900, 1993.

Shelling and Smith, Gene Therapy, 1:165-169, 1994.

Steinhelper et al., Am. J. Physiol. 259 (6 Pt 2), H1826-34, 1990.

Stewart et al., EMBO J. 6:383-388, 1987.

Stratford-Perricaudet and Perricaudet, In: Human Gene Transfer, Eds, O. Cohen-Haguenauer and M. Boiron, Editions John Libbey Eurotext, France, pp. 51-61, 1991.

Stratford-Perricaudet et al., Hum. Gene. Ther., 1:241-256, 1990.

Tedesco et al., J. Virol. 6947-6957, 1995.

Temin, In: Gene Transfer, Kucherlapati (ed.), New York: Plenum Press, pp. 149-188, 1986.

Top et al., J. Infect. Dis., 124:155-160, 1971.

Tratschin et al., Mol. Cell. Biol., 4:2072-2081, 1984.

Tratschin et al., Mol. Cell Biol., 5:3258-3260, 1985.

Tur-Kaspa et al., Mol. Cell Biol., 6:716-718, 1986.

Van der Putten et al., Proc. Nat'l Acad. Sci. USA 82:6148-6152, 1985.

Wagner et al., Proc. Nat'l Acad. Sci. USA 87(9):3410-3414, 1990.

Walsh et al., J. Clin. Invest, 94:1440-1448, 1994.

Wei et al., Gene Therapy, 1:261-268, 1994.

Wong et al., Gene, 10:87-94, 1980.

Wu and Wu, J. Biol. Chem., 262:4429-4432, 1987.

Wu and Wu, Biochemistry, 27:887-892, 1988.

Wu and Wu, Adv. Drug Delivery Rev., 12:159-167, 1993.

Yamauchi-Takihara, et. al., Proc. Nat'l Acad. Sci. USA 86(10):3504-3508, 1989.

Yang et al., Proc Nat'l Acad. Sci. USA, 87:9568-9572, 1990.

Yang et al., Proc. Nat'l Acad. Sci. USA, 91:4407-4411, 1994.

Yoder et al., Blood, 82 (Supp.): 1:347A, 1994.

Zhou et al., Exp. Hematol, 21:928-933, 1993.

Zhou et al., J. Exp. Med., 179:1867-1875,1994.

Ziober and Kramer, J. Bio. Chem., 271(37):22915-22, 1996

What is claimed is:

1. A transgenic mouse, cells of which comprise an expression cassette comprising a tissue selective promoter operably linked to a nucleic acid segment encoding SV40 large T antigen, wherein said nucleic acid segment is flanked 5′ and 3′ by site specific excision sequences.

2. The mouse of claim 1, wherein said tissue selective promoter is preferentially active in cardiac cells.

3. The mouse of claim 2, wherein said cardiac tissue selective promoter is Nkx2.5.

4. The mouse of claim 1, wherein said site specific excision sequences are loxP sites.

5. The mouse of claim 1, wherein said expression cassette further comprises a selectable or screenable marker.

6. A method for obtaining a transgenic murine progenitor cell line comprising:
(a) transforming one or more murine embryonic cells with an expression cassette comprising a tissue selective promoter operably linked to a nucleic acid segment encoding SV40 large T antigen, wherein said nucleic acid segment is flanked 5′ and 3′ by site specific excision sequences;
(b) inserting said one or more murine embryonic cells into a surrogate mouse mother;
(c) obtaining one or more pups from said surrogate mouse mother;
(d) identifying one or more pups that express SV40 large T antigen in a tissue selective manner; and
(e) obtaining cells from said one or more pups that express SV40 large T antigen.

7. The method of claim 6, wherein said tissue selective promoter is preferentially active in cardiac cells.

8. The method of claim 7, wherein said cardiac tissue selective promoter is Nkx2.5.

9. The method of claim 6, wherein said site specific excision sequences are loxP sites.

10. The method of claim 6, wherein said expression cassette further comprises a selectable or screenable marker.

11. The method of claim 6, further comprising the step of activating site specific excision, thereby eliminating said nucleic acid segment encoding SV40 large T antigen.

12. The method of claim 11, wherein activating site specific excision comprises transforming cells of step (e) with an expression construct comprising a promoter operably linked to a nucleic acid segment encoding Cre protein.

13. The method of claim 12, wherein said expression construct is a viral expression construct.

14. The method of claim 13, wherein said viral expression construct is adenovirus.

15. The method of claim 12, wherein said promoter is a constitutive promoter.

16. The method of claim 12, wherein said promoter is a tissue selective promoter.

17. A transgenic murine progenitor cell line comprising an expression cassette comprising a tissue selective promoter operably linked to a nucleic acid segment encoding SV40 large T antigen, wherein said nucleic acid segment is flanked 5′ and 3′ by site specific excision sequences.

18. The murine progenitor cell line of claim 17, wherein said tissue selective promoter is preferentially active in cardiac cells.

19. The murine progenitor cell line of claim 18, wherein said cardiac tissue selective promoter is Nkx2.5.

20. The murine progenitor cell line of claim 17, wherein said site specific excision sequences are loxP sites.

21. The murine progenitor cell line of claim 17, wherein said expression cassette further comprises a selectable or screenable marker.

22. The murine progenitor cell line of claim 17, wherein said cell line is derived from cells of liver, neuronal, glial, skeletal satellite, cardiac or erythroid tissue.
